(19) United States
(12) Patent Application Publication
(10) Pub. No.: US 2002/0161007 A1
(43) Pub. Date: Oct. 31, 2002

(54) Non-steroidal modulators of estrogen receptors

(76) Inventors: Mary Jane Meegan, Dublin (IE); Rosario Bridget Hughes, Bansha (IE); Daniela Maria Zisterer, Dublin (IE); David George Lloyd, Dublin (IE)

Correspondence Address: JACOBSON HOLMAN PLLC, 400 SEVENTH STREET N.W., SUITE 600, WASHINGTON, DC, 20004, US

(21) Appl. No.: 10/077,951

(22) Filed: Feb. 20, 2002

(30) Foreign Application Priority Data
Feb. 20, 2001 (IE) 2001/0156

(51) Int. Cl. C07D 265/32; A61K 31/535; A61K 31/445; A61K 31/137

(52) U.S. Cl. 514/239.2; 544/170; 548/215; 548/570; 564/346; 564/367; 514/317; 514/374; 514/651; 514/408

(57) ABSTRACT

Estrogen receptor modulators, compositions comprising the compounds and methods relating to the use thereof are described. The compounds may be used in inhibiting the proliferation of and/or induces apoptosis in human breast cancer cells.

The invention relates to compounds which function as modulators of estrogen receptors and which induce apoptosis in certain cell lines and pharmaceutical compositions containing the compounds.

### GENERAL BACKGROUND

The estrogen receptor is responsible, among other functions, as a ligand-inducible nuclear transcription factor, for the mediation of the physiological effects of estrogen steroid hormones [1]. Through binding to the ligand-binding domain of the receptor, hormone ligands initiate a cascade of molecular and biochemical events which ultimately can express themselves in the growth of certain tissues through the activation or inactivation of particular genes [2]. Non-steroidal antiestrogens, by definition, antagonise the activity of estrogenic species. One such compound is tamoxifen ((Z)-1-[4-(2-dimethylaminoethoxy)-phenyl]-1,2-diphenyl-1-butene), which has been used extensively in the treatment of hormone-sensitive breast cancers, and has become the first-line endocrine therapy for all stages of breast cancer in pre-and post-menopausal women [3]. Now classified as a selective estrogen receptor modulator (SERM) by virtue of its estrogen-like effects in certain tissues, the antiestrogenic properties of this compound are related to its ability to compete for estrogen binding sites in target tissues such as the breast. In very general terms, the estrogen receptors (ER) function as follows upon receipt/binding of a suitable ligand to the ligand binding domain (LBD). Agonist (estrogen) binding to the LBD causes receptor dissociation from its location in heat-shock protein, dimerisation and eventual transcription. The physical binding of a ligand initiates a conformational change in the receptor. If the ligand is an agonist, the receptor folds in such a way that helix 12 (H 12) of the protein closes tightly over the top of the ligand binding domain, and this folding forms a hydrophobic cavity or cleft on the surface of the protein. This cleft acts as a nuclear receptor coactivator binding site on the surface and facilitates nuclear transcription by the receptor [4]. Contrarily, on binding an antagonist, H12 can no longer fold over the LBD and the coactivator binding site is not formed, thus preventing the receptor from fulfilling its role in transcription. The ER should not be thought of as the controller of gene transcription but rather as a choreographer in the transcription ballet. The action of a compound as a selective estrogen receptor modulator may be rationalised as follows. Different modulators will interact with the ligand binding domain of an estrogen receptor in differing ways so as to subtly affect not only the orientation of the region identified as H12 within the ligand binding domain of the receptor, but the overall bound conformation of the entire receptor. Such conformational changes and differences in receptor-ligand complexes inherently affect the coactivator binding sites of the receptor. This affection of coactivator binding sites influences the manner and degree to which the receptor will function in its transcription role—the potential for different levels of estrogenic or antiestrogenic activity may be rationalised—the basis of SERM action.

It was thought until quite recently that these physiological effects arose through the influence of a single receptor. The discovery of a second receptor subtype, resulted in the classification of two isoforms—the ERα and ERβ [5]. Even more recent is the discovery of perhaps another variant of isoform ERβ-ERβ2-with significant variation in its ligand-binding domain. It is thought that this ‘new’ isoform may function as a negative regulator of estrogen action [6]. The ERα dominates in reproductive tissues such as the uterus and breast, whereas ERβ has a diverse tissue distribution, being expressed in the central nervous system, the gastrointestinal tract, the kidneys and the lungs—it is the β form which predominates in the ovaries however. Both ERα and ERβ are found in breast tissue, with the alpha isoform apparently playing the more important role [7].

Compounds which modulate the ER, as either agonists or antagonists, or in a tissue selective manner are recognised for their pharmaceutical utility in the treatment of a wide variety of estrogen-related conditions, including conditions related to the central nervous system, skeletal system, reproductive system, cardiovascular system, skin, hair follicles, immune system, bladder and prostrate as well as estrogen receptor-and non-estrogen receptor-expressing tumors. In addition to such estrogen related conditions, some estrogen receptor modulators have been shown to inhibit the proliferation of certain cell-lines not only through estrogen antagonism, but also, through the sustained induction of programmed cell death, apoptosis [8, 9]. Apoptotic cell death can be induced by a variety of drugs with diverse chemical structures and different mechanisms of action. Among the list of apoptosis-inducing agents are a wide range of anti-cancer drugs. Given the importance of the estrogen receptor and the potential application of modulators in so many disease processes the design of therapeutics which modulate this target continues to generate considerable interest both industrial and academic [10]. It has been suggested that building flexibility into the rigid backbone of antiestrogens could enhance their activity and binding affinity for the estrogen receptor [11].

Accordingly there is a general need in the art for effective estrogen receptor modulators, and more specifically for potent compounds which possess a degree of molecular flexibility and can demonstrate positive induction of apoptosis in key cell lines, including pharmaceutical compositions comprising such compounds as well as methods relating to the use thereof.

Statements of Invention

According to the invention there is provided a compound of the formula I

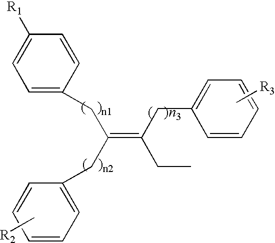

wherein R₁=H, OH, Br, NH₂ or R₄ wherein R₄ is O(CH₂)₂NRₐR_{b} or NH(CH₂)ₓNRₐR_{b} or NH(CH₂)ₓRₐ-R_{b} or O(CH₂)ₓRₐ-R_{b} and Rₐ and R_{b} are independently H, O, CH₃, C₂ H₅, C₃ H₇ or optionally part of a heterocyclic

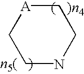

ring system of the structure:

wherein n₄ and n₅ are independently 0 or 1 and both are not 0, and

A is CH₂ or O; and

x is 2 or 3,

R₂ is independently one of H, OH, OPiv, OAc, OCONHMe, OMe R₃ is independently one of H, OH, OPiv OMe or para O(CH₂)₂NRₐR_{b} wherein Rₐ and R_{b} are as defined above,

n₁, n₂ and n₃=0 or 1 independently, and n₁, n₂ and n₃ are such that only one n=1 at any one time where n₁, n₂ and n₃ are not all equal to 0,

and isomers, prodrugs and pharmaceutically acceptable salts thereof.

Preferably R₂ is not the same as R₃.

Variation in the hydroxyl protecting groups provides enhanced potential metabolic profiles for these compounds.

The invention also provides a compound having the formula

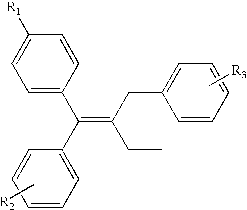

wherein

R₁ is as defined above

R₂ is independently one of H, OH, OPiv, OAc, OCONHMe,

OMe

R₃ is independently one of H, OH, OPiv, OMe,

and isomers, prodrugs and pharmaceutically acceptable salts thereof.

The invention also provides a compound having the formula

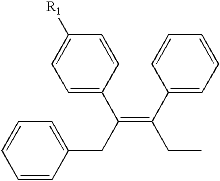

wherein R₁=H, OH, Br, NH₂ or R₄ wherein R₄ is H, O(CH₂)₂NRₐR_{b} or NH(CH₂)ₓNRₐR_{b} or NH(CH₂)ₓRₐ-R_{b} or O(CH₂)ₓRₐ-R_{b} and Rₐ and R_{b} are independently H, O, CH₃, C₂ H₅, C₃ H₇ or optionally part of a heterocyclic ring system of the structure:

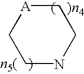

wherein n₄ and n₅ are independently 0 or 1 and both are not 0,

A is CH₂ or O,

and x is 2 or 3,

and isomers, prodrugs and pharmaceutically acceptable salts thereof.

The invention further provides a compound having the formula

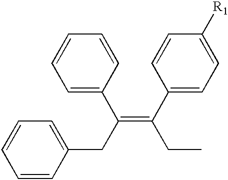

wherein R₁=H, OH, Br, NH₂ or R₄ wherein R₄ is H, O(CH₂)₂NRₐR_{b} or NH(CH₂)ₓNRₐR_{b} or NH(CH₂)ₓRₐ-R_{b} or O(CH₂)ₓRₐ-R_{b} and Rₐ and R_{b} are independently H, O, CH₃, C₂ H₅, C₃ H₇ or optionally part of a heterocyclic ring system of the

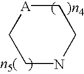

structure:

wherein n₄ and n₅ are independently 0 or 1 and both are not 0,

A is CH₂ or O,

and x is 2 or 3,

The invention further provides a compound having the formula

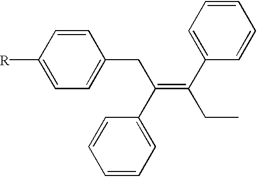

wherein R=H, OH, Br, NH₂ or R₄ wherein R₄ is O(CH₂)₂NRₐR_{b} or NH(CH₂)ₓNRₐR_{b} or NH(CH₂)ₓRₐ-R_{b} or O(CH₂)ₓRₐ-R_{b} and Rₐ and R_{b} are independently H, O, CH₃, C₂ H₅ , C₃ H₇ or optionally part of a heterocyclic ring system of the structure:

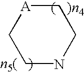

wherein n₄ and n₅ are independently 0 or 1 and both are not 0,

A is CH₂ or O,

and x is 2 or 3,

and isomers, prodrugs and pharmaceutically acceptable salts thereof.

The invention further provides a compound of the formula

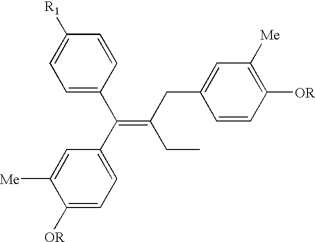

wherein R=H, Me or Piv and wherein R₁=H, Br, NH₂ or R₄ wherein R₄ is O(CH₂)₂NRₐR_{b} or NH(CH₂)ₓNRₐR_{b} or NH(CH₂)ₓRₐ-R_{b} or O(CH₂)ₓRₐ-R_{b} and Rₐ and R_{b} are independently H, O, CH₃, C₂ H₅, C₃ H₇ or optionally part of a heterocyclic ring system of the structure:

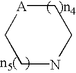

wherein n₄ and n₅ are independently 0 or 1 and both are not 0,

A is CH₂ or O,

x is 2 or 3,

and isomers, prodrugs and pharmaceutically acceptable salts thereof.

The compounds of the invention have inherent flexibility which provides beneficial binding properties in the ER and also have a modified electronic conjugation which can aid against the formation of metabolic carbocationic intermediates and ultimate DNA adduct formation and hepatcarcinogenicity.

The flexibility derives from the presence of an additional methylene group which is not present in other known SERM compounds.

Preferably R₁ is O(CH₂)₂Rₐ-R_{b} and Rₐ and R_{b} are as defined hereinbefore.

Also preferred are compounds in which n₃ is 1 and Rₐ-R_{b} are selected from pyrrolidinyl or piperidyl. These compounds are preferred because of their anti-proliferative effect on breast tumour cells.

Preferably the invention provides a compound wherein at least one or both of R₂ or R₃ contains an oxygen group. Preferably R₂ or R₃ may be in any position on the associated ring.

Preferably the invention provides a compound wherein R₂ is a para hydroxy group. In this case R₃ is preferably hydrogen. These compounds are preferred because of their anti-proliferative effect on breast tumour cells.

From studies of the binding mode of ligands within the LBD of estrogen receptors, compounds containing hydroxy substituents, particularly para-, mono and di-hydroxy containing compounds, were found to interact strongly with glutamine, arginine and histidine amino acid residues responsible for ligand anchoring within the active site. The di-hydroxy compounds were found to exhibit high antiproliferative potencies and increased ER binding affinity.

In one embodiment the invention provides a compound wherein R₂ is an ester group in the para position. In this case preferably R₃ is hydrogen.

Preferred compounds of the invention are those having a hydroxyl or ester group in the para position of R₂. These compounds are preferred because they show good anti-proliferative effect on the human breast tumour MCF-7 cell line.

In one embodiment the invention provides a compound wherein R₃ is an ortho methoxy group.

Most preferably R₁ is O(CH₂)₂Rₐ-R_{b} and Rₐ-R_{b} is morpholinyl.

In one embodiment the invention R₁ is O(CH₂)₂Rₐ-R_{b} and Rₐ-R_{b} is pyrrolidinyl.

Preferably a compound of the invention is selected from

2-Benzyl-1-phenyl-1-[4-(dimethylaminoethoxy)phenyl]but-1-ene

2-Benzyl-1-phenyl-1-[4-(diethylaminoethoxy)phenyl]but-1-ene

2-Benzyl-1-phenyl-1-[4-(pyrrolidinylethoxy)phenyl]but-1-ene

2-Benzyl-1-phenyl-1-[4-(pipyridinylethoxy)phenyl]but-1-ene

2-Benzyl-1-phenyl-1-[4-(morpholinylethoxy)phenyl]but-1-ene

1-Benzyl-2-phenyl-[(4-dimethyleaminoethoxy)phenyl]but-1-ene

1-Benzyl-2-phenyl-[1-(4-diethylaminoethoxy)phenyl]-but-1-ene

1-Benzyl-2-phenyl-[1-(4-pyrrolidinylethoxy)phenyl]-but-1-ene

1-Benzyl-2-phenyl-[1-(4-pipyridinylethoxy)phenyl]-but-1-ene

1-Benzyl-2-phenyl[1-(5-morpholinylethoxy)phenyl]but-1-ene

1-Benzyl-1-phenyl-2-[(4-dimethylaminoethoxy)phenyl]but-1-ene

1-Benzyl-1-phenyl-2-[(4-diethylaminoethoxy)phenyl]but-1-ene

1-Benzyl-1-phenyl-2-[(4-pyrrolidinylethoxy)phenyl]but-1-ene

1-Benzyl-1-phenyl-2-[(4-pipyridinylethoxy)phenyl]but-1-ene

1-Benzyl-1-phenyl-2-[(4-morpholinylethoxy)phenyl]but-1-ene

1,2-Diphenyl-2-[2-(dimethylaminoethoxy)benzyl]but-1-ene

1,2-Diphenyl-2-[2-(diethylaminoethoxy)benzyl]but-1-ene

1,2-Diphenyl-2-[(4-pyrrolidinylethoxy)benzyl]but-1-ene

1,2-Diphenyl-2-[(4-pipyridinylethoxy)benzyl]but-1-ene

1,2-Diphenyl-2-[(4-morpholinylethoxy)benzyl]but-1-ene

2,2-Dimethyl-propionic acid 4-{2-benzyl-1-[4-(2-pyrrolidin-1-yl-ethoxy-phenyl]-but-1-enyl}-phenyl ester

2,2-Dimethyl-propionic acid 4-{2-benzyl-1-[4-(2-dimethylamino-ethoxy-phenyl]-but-1-enyl}-phenyl ester

2,2-Dimethyl-propionic acid 4-{2-benzyl-1-[4-(2-morpholin-4-yl -ethoxy-phenyl]-but-1-enyl}-phenyl ester

4-{2-Benzyl-1-[4-(2-dimethylamino-ethoxy)-phenyl]-but-1-enyl}-phenol

4-{2-Benzyl-1-[4-(2-pyrrolidin-1-yl-ethoxy)-phenyl]-but-1-enyl}-phenol

2,2-Dimethyl-propionic acid 4-{2-(2-methoxybenzyl)-1-[4-(2-pyrrolidin-1-yl-ethoxy)-phenyl]-but-1-enyl}-phenyl ester

[4-(2-Benzyl-1-phenylbut-1-enyl)-phenyl]-(2-methoxyethyl)-amine

[4-(2-Benzyl-1-phenylbut-1-enyl)-phenyl]-(2-ethoxyethyl)-amine

[4-(2-Benzyl-1-phenylbut-1-enyl)-phenyl]-(2-propoxyethyl)-amine

[4-(2-Benzyl-1-phenylbut-1-enyl)-phenyl]-(3-methoxypropyl)-amine

[4-(2-Benzyl-1-phenylbut-1-enyl)-phenyl]-(3-ethoxypropyl)-amine

[4-(2-Benzyl-1-phenylbut-1-enyl)-phenyl]-(3-propoxypropyl)-amine

2-[4-(2-Benzyl-1-phenylbut-1-enyl)-phenylamino]-ethanol

3-[4-(2-Benzyl-1-phenylbut-1-enyl)-phenylamino]-propan-1-ol

2-[4-(2-Benzyl-1-phenylbut-1 -enyl) -phenoxy]-ethanol

3-[4-(2-Benzyl-1-phenylbut-1-enyl)-phenoxy]-propan-1-ol

{4-[2-Benzyl-1 -(4-methoxyphenyl)-but-1 -enyl]-phenyl}-(2-methoxyethyl)-amine

{4-[2-(4-methoxybenzyl)-1-(4-methoxyphenyl)-but-1-enyl]-phenyl}-(2-methoxyethyl)-amine

{4-[2-(4-methoxybenzyl)-1-phenylbut-1-enyl]-phenyl}-(2-methoxyethyl)-amine

1-(2-Benzyl-1-phenylbut-1-enyl)-4-(2-methoxyethoxy)-benzene

4-{2-Ethyl-3-[4-(2-methoxyethylamino)-phenyl]-3-phenylallyl}-phenol

N′-[4-(2-Benzyl-1-phenylbut-1-enyl)-phenyl]-N,N-dimethylethane-1,2-diamine.

In one embodiment the invention provides a compound wherein the compound is antiosteoporotic.

In another embodiment the invention provides a compound wherein the compound inhibits the proliferation of and/or induces apoptosis in human breast cancer cells.

Preferably the invention provides a compound wherein the compound is a modulator of the estrogen receptor(s).

The invention also provides compounds of formula I and isomers thereof for use as intermediates in the synthesis of other compounds of formula I. Some of the preferred intermediate compounds are selected from

4-(2-benzyl-1-phenylbut-1-enyl)phenyl-amine

N-[4-(2-benzyl-1-phenylbut-1-enyl)-phenyl]-2,2,2-trifluoroacetamide

N-[4-(2-benzyl-1-phenylbut-1-enyl)-phenyl]-2,2,2-trifluoro-N-[3-(tetrahydropyran-2-yloxy)-propyl]-acetamide

[4-(2-Benzyl-1-phenylbut-1-enyl)-phenyl]-[3-(tetrahydropyran-2-yloxy)-propyl]-amine

[4-(2-benzyl-1-phenylbut-1-enyl)-phenyl]-[3-(tetrahydropyran-2-yloxy)-propyl]-carbamic acid ethyl ester

[4-(2-benzyl-1-phenylbut-1-enyl)-phenyl]-(3-hydroxypropyl)-carbamic acid ethyl ester

The invention further provides a pharmaceutical composition comprising a compound of the invention. Preferably the pharmaceutical composition is in combination with a pharmaceutically acceptable carrier or diluent. Most preferably in combination with a pharmaceutically active compound.

Preferably the pharmaceutically active compound is an anti-cancer drug, most preferably cisplatin.

The pharmaceutical composition of the invention may be administered in the form of an emulsion, liposome, patch, powder and/or complex tablet, capsule, syrup, dose-metered inhaler.

Preferably the pharmaceutical composition is in a form for oral, intravenous, intramuscular, intraperitoneal, intradermal, intravesicular and/or rectal administration.

In one embodiment the invention provides a pharmaceutical composition comprising a compound of the invention for use in the preparation of a medicament for the prophylaxis and/or treatment of estrogen related conditions and/or conditions where the induction of apoptosis is desirable. Preferably the condition is any one or more of obesity, hormone dependent breast cancer, osteoporosis, estrogen deficiency, arthritis, cardiovascular disease, ovarian cancer, artherosclerosis, colon tumor, endometriosis, Alzheimer's disease, non-insulin dependent (type II) diabetes, infertility, prostrate tumor, melanoma, acne, hypercholesterolemia, CNS disease, contraception, conditions related to hair follicles, macular degeneration, urinary incontinence, estrogen receptor-expressing and estrogen receptor-expressing tumors, leukaemia.

In one embodiment the invention provides use of a compound of the invention in inhibiting the proliferation of and/or induction of apoptosis in breast cancer cells.

The invention also provides a compound of the invention for the preparation of a medicament for use in the prophylaxis and/or treatment of an estrogen related disease. Most preferably in the prophylaxis and/or treatment of breast cancer.

The invention further provides a method for the treatment and/or prophylaxis of an estrogen related disease comprising administering an effective amount of a compound of the invention or a composition of the invention.

### DETAILED DESCRIPTION

The present invention is directed to estrogen receptor modulators (encompassing antagonists and/or agonists), pharmaceutical compositions comprising such modulators and their use in methods for treating estrogen related conditions and conditions wherein the induction of apoptosis is desirable. Such conditions are discussed in detail below, and generally include (but are not limited to) obesity, hormone dependent breast cancer, osteoporosis, estrogen deficiency, arthritis, cardiovascular disease, ovarian cancer, artherosclerosis, colon tumor, endometriosis, Alzheimers disease, non-insulin dependent (type II) diabetes, infertility, prostrate tumor, melanoma, acne, hypercholesterolemia, CNS disease, contraception, conditions related to hair follicles, macular degeneration, urinary incontinence, estrogen receptor-expressing and non-estrogen receptor-expressing tumors, leukaemia.

Throughout the specification the term estrogen agonist refers to a compound that binds to an estrogen receptor (ER) and mimics the action of estrogen in one or more tissues. An antagonist binds to ER and blocks the action of estrogen in one or more tissues.

The compounds of the invention have the following general structures which are grouped under generic types for ease of description.

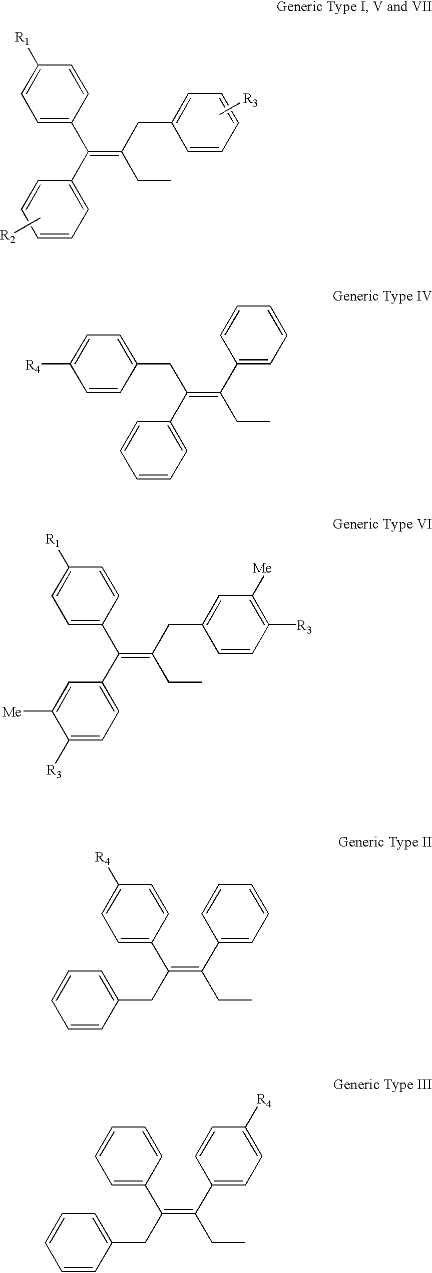

wherein R₁=H or OH or Br or NH₂ or R₄ wherein R₄ is O(CH₂)₂NRₐR_{b} or

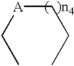

NH(CH₂)ₓNRₐR_{b} or NH(CH₂)ₓRₐ-R_{b} or O(CH₂)ₓRₐ-R_{b} and Rₐ and R_{b} are independently H, O, CH₃, C₂ H₅, C₃ H₇ or optionally part of a heterocyclic ring system of the structure:

wherein n₄ and n₅ are independently 0 or 1 and both are not 0.

A is CH₂ or O

x is 2 or 3

R₂ is independently one of H, OH, OPiv, OAc, OCONHMe ,OMe

R₃ is independently one of H, OH, OPiv, OMe.

The invention includes stereoisomers, geometric isomers, prodrugs and pharmaceutically acceptable salts of the compounds.

The utility of these compounds in any of the cited disease states or conditions would involve the administration of an effective amount of a compound of this invention, preferably in the form of a pharmaceutical composition to an animal in need thereof, including a human.

The methods of the invention include administration of an effective amount of a compound of the invention, or a salt thereof as the active ingredient. Pharmaceutically acceptable salts are typically salts of non-toxic type commonly used, such as salts with organic acids, inorganic acids and amino acids. These salts may be prepared by the methods known to chemists of ordinary skill.

The compounds of the invention may be administered to animals (including humans) orally or parenterally in the conventional form of preparation such as capsules, microcapsules, tablets, granules, powder, troches, pills, suppositories, injections, suspensions and syrups. Suitable formulations may be prepared by methods commonly employed using conventional organic and inorganic additives such as an excipient, a binder, a disintegrator, a lubricant, a flavouring agent, a preservative, a stabiliser, a suspending agent, a dispersing agent, a diluent and base wax. The amount of active ingredient in the medical composition may be at a level that will exercise the desired therapeutic effect.

Pharmaceutical chemists will recognise that physiologically active compounds containing one or more accessible hydroxyl moieties are frequently administered in the form of pharmaceutically acceptable esters or as prodrugs, which is well documented in prior literature [12]. Prodrugs are covalently bound carriers that release a parent compound in vivo (believed to be mediated through metabolism), and would include for example compounds of the invention wherein accessible hydroxy groups were bonded to any group that, when administered to a patient, cleaves or is metabolised to form the hydroxy group. It is known in the pharmaceutical field to adjust the rate or duration of action of a compound by appropriate choices of such covalently bound groups. To this end, prodrugs are also included within the context of the invention.

The compounds of this invention may be made by one skilled in organic synthesis by known techniques, as well as by the synthetic routes disclosed hereafter. For example, a general reaction scheme for the formation of representative compounds of the invention is as follows:

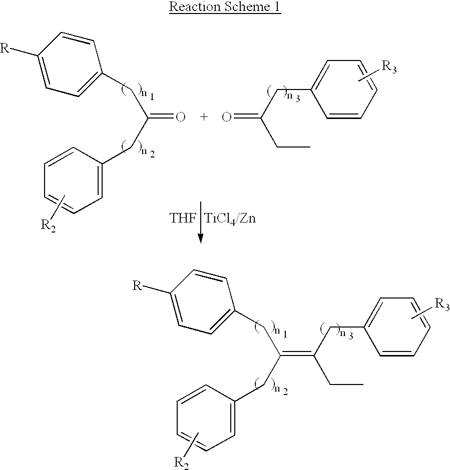

wherein R is selected from H, OH, NH₂ or is R₄=O(CH₂)₂NRₐR_{b} or NH(CH₂)ₓNRₐR_{b} or NH(CH₂)ₓRₐ-R_{b} or O(CH₂)ₓRₐ-R_{b} and RₐR_{b} are as detailed previously and R₄ is introduced as R through standard chemical transformations starting from when R=OH or NH₂ R₂ is selected from H, OH, OPiv, OAc, OCONHMe, OMe R₃ is selected from H, OH, OPiv, OMe, or is R₄=para-O(CH₂)₂NRₐR_{b} and RₐR_{b} are as detailed previously and R₄ is introduced as R3 through standard chemical transformation starting from when R₃=para-OH. n₁, n₂ and n₃=0 or 1 independently, and n₁, n₂ and n₃ are such that only one n=1 at any one time to reflect structures given in the summary and detailed descriptions above and where n₁, n₂ and n₃ do not all together=0

The nature of the titanium coupling reaction is such that isomeric E & Z mixtures may be produced during synthesis, which may be separated into single isomers through known preparative analytical and chemical techniques. All such isomeric forms are included within the present invention, including mixtures thereof. Furthermore some of the compounds may form polymorphic crystalline entities or solvates with water or other organic solvents and these compound forms are similarly included in this invention.

Examples 1 to 13 describe the synthesis of representative compounds of the invention.

EXAMPLE 1—Formation of 2-Benzyl-1-phenyl-1-[4-(dimethylaminoethoxy) phenyl]but-1-ene (Generic Type I)

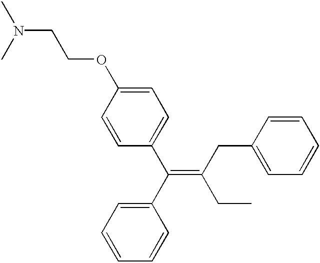

4-(Dimethylaminoethoxy)benzophenone (1.20 g, 4.5 mmol) was placed in a three-necked round bottomed flask equipped with a magnetic stirrer. To this 1-phenyl-2-butanone (0.662 ml, 4.5 mmol) and absolute dioxane (25 ml) were added and the mixture stirred in an ice bath (0-5° C., 15 min). Titanium tetrachloride (0.99 ml, 9.1 mmol) was slowly added via syringe over 10 min, while maintaining stirring and the lowered temperature. Upon completion of addition the reaction mixture was left stirring for a further 30 min, after which time Zn powder ((1.86 g, 28 mmol), particle size<10 micron) was added in a single portion and stirring continued for 15 min. The ice bath was removed and the reaction mixture allowed to reach room temperature, at which stage the apparatus was arranged for reflux and the reaction brought to reflux temperature for 4 h. The reaction was allowed to cool to room temperature, filtered (residue washed with ethyl acetate), washed, first with 10% K₂CO₃ solution, then a large volume of deionised water and extracted (3×30 ml) into dichloromethane. The organic extracts were combined and consecutively washed with 20 ml 3 M HCI and deionised water before being dried over anhydrous sodium sulphate. The resulting solution was filtered to remove drying agent and concentrated under reduced pressure rotary evaporation. The crude product was purified using column chromatography CH₂Cl₂/MeOH (60:40) to yield the pure target compound (0.41 g, 23%). The crude product was purified using column chromatography CH₂Cl₂/MeOH (60:40) to yield pure product (0.41 g, 23%—(product homogenous on TLC with rf=0.32; 60/40 CH₂Cl₂/Pet. Ether)). HPLC RT=15.0, 16.2 mins. ¹ H-NMR (CDCl₃, 400 MHz) δ=0.99 (m, 3 H, CH₃), 2.07 (m, 2 H, CH₂), 2.48 (d, 6 H, J=7.95, (CH₃)₂), 2.90 (m, 2 H, NCH₂), 3.56 and 3.59 (2×s, 2 H, CH₂), 4.16 (m, 2 H, OCH₂), 6.82-6.84 (d, 2 H, J=8.74, Ar), 7.1-7.32 (m, 12 H, Ar) ppm. ¹³C-NMR (CDCl₃, 76.7 MHz)δ=12.81 (CH₃), 24.20 (CH₂), 36.72 (CH₂), 44.75 (N(CH₃)₂), 57.34 (CH₂N), 64.67 (OCH₂), 113.67-140.19 (Ar C) ppm. IR (film) v=3007.5, 2997.8, 1606.6 (C=C), 1506.5, 1461.5, 1371.4, 1275.7, 1173.4 cm⁻¹; HRMS calcd. 385.2405, found 385.2406.

Further compounds of this generic type given below were prepared by analogous methods. Further details are given in Appendix 1.

2-Benzyl-1-phenyl-1-[4-(diethylaminoethoxy)phenyl]but-1-ene

The pure product was isolated in 40% yield following flash chromatography (CH₂Cl₂/MeOH (75:25)—(product homogenous on TLC with rf=0.45; 60/40 MeOH/CH₂Cl₂)). HPLC RT=18.0, 19.5 mins. ¹ H-NMR (CDCl_{3, 400} MHz)δ=0.95 (m, 3 H, CH₃), 2.05 (m, 2 H, CH₂), 2.51 (m, 5 H, (CH₂CH₃)), 2.71 (m, 5 H, (CH₂CH₃)), 2.91 (m, 2 H, NCH₂), 3.60 and 3.64 (2×s, 2 H, CH₂), 4.10 (m, 2 H, OCH₂), 6.80-6.86 (d, 2 H, J=8.34, Ar), 7.18-7.41 (m, 12 H, Ar) ppm. ¹³C-NMR (CDCl₃, 76.7 MHz)δ=11.14 (CH₃), 24.30 (CH₂), 36.86 (CH₂), 47.33 (N(CH₂)₂), 51.20 (CH₂N), 65.15 (OCH₂), 113.60−135.20 (Ar C) ppm. IR (film) v=3027.2, 2969.4, 1600.0 (C=C), 1508.0, 1454.0, 1243.7, 1175.8 cm⁻¹; HRMS calcd. 413.2725, found 413.2719.

2-Benzyl-1-phenyl-1-[4-(pyrrolidinylethoxy)phenyl]but-1-ene

The pure product was isolated in 40% yield following flash chromatography (CH₂Cl₂/MeOH (90:10)—(product homogenous on TLC with rf=0.49; 60/40 MeOH/CH₂Cl₂)). HPLC RT=21.0, 22.2 mins. ¹ H-NMR (CDCl₃, 400 MHz)δ=1.02 (m, 3 H, CH₃), 1.84 (m, 4 H, (CH₂)₂), 2.12 (m, 2 H, CH₂), 2.71 (m, 4 H, (CH₂)₂), 2.94 (m, 2 H, NCH₂), 3.63 and 3.67 (2 ×s, 2 H, CH₂), 4.13 (m, 2 H, OCH₂), 6.85-6.95 (d, 2 H, J=8.04, Ar), 7.21-7.38 (m, 12 H, Ar) ppm. ¹³C-NMR (CDCl₃, 76.7 MHz)δ=12.93 (CH₃), 23.50 (CH₂), 24.79 (CH₂), 37.25 (CH₂), 54.60 (N(CH₂)₂), 55.01 (CH₂N), 66.77 (OCH₂), 113.84-138.18 (Ar C) ppm. IR (film) v=3026.2, 2964.3, 1708.6 (C=C), 1507.5, 1454.0, 1243.8, 1175.1 cm⁻¹; HRMS calcd. 411.2573, found 411.2562.

2-Benzyl-1-phenyl-1-[4-(pipyridinylethoxy)phenyl]but-1-ene

The pure product was isolated in 24% yield following flash chromatography (CH₂Cl₂/MeOH (70:30)—(product homogenous on TLC with rf=0.49; 60/40 MeOH/CH₂Cl₂)). HPLC RT=15.0, 16.2 mins.¹ H-NMR (CDCl₃, 400 MHz) δ=0.97 (m, 3 H, CH₃), 1.47 (m, 2 H, CH₂), 1.62 (m, 4 H (CH₂)₂), 2.03 (m, 2 H, CH₂), 2.49 (m, 4 H, (CH₂)₂), 2.81 (m, 2 H, NCH₂), 3.57 and 3.61 (2×s, 2 H, 15.04, CH₂), 4.09 (m, 2 H, OCH₂), 6.82-6.88 (d, 2 H, J=8.52, Ar), 7.15-7.17 (m, 12 H, Ar) ppm. ¹³C-NMR (CDCl₃, 76.7 MHz) δ=12.81 (CH3), 23.66 (CH₂), 24.29 (CH₂), 25.36 (CH₂), 36.85 (CH₂), 54.54 (N(CH₂)₂), 57.48 (CH₂N), 65.30 (OCH₂), 113.76-138.13 (Ar C) ppm. IR (film) v=3026.1, 2932.8, 1708.6 (C=C), 1506.8, 1453.2, 1241.7, 1174.9 cm⁻¹; HRMS calcd. 425.2718, found 425.2719.

2-Benzyl-1-phenyl-1-[4-(morpholinylethoxy)phenyl]but-1-ene

The pure product was isolated in 34% yield following flash chromatography (CH₂Cl₂/MeOH (90:10)—(product homogenous on TLC with rf=0.49; 90/10 Pet. Ether/EtOAc)). HPLC RT=22.8, 24.0 mins.¹ H-NMR (CDCl₃, 400 MHz) δ=0.95 (m, 3 H, CH₃), 2.04 (m, 2 H, CH₂), 2.58 (m, 4 H (CH₂)₂), 2.81 (m, 2 H, NCH₂), 3.56 and 3.59 (2×s, 2 H, CH₂), 3.71 (m, 4 H, (CH₂)₂), 4.07 (m, 2 H, OCH₂), 6.82-6.87 (d, 2 H, J=8.52, Ar), 7.15-7.29 (m, 12 H, Ar) ppm. ¹³C-NMR (CDCl₃, 76.7 MHz) δ=13.20 (CH₃), 37.14 (CH₂), 54.03 (NCH₂), 57.65 (CH₂), 65.66 (OCH₂), 66.87 (CH₂), 114.16-138.53 (Ar C) ppm. IR (film) v=3026.8, 2963.1, 1712.6 (C=C), 1507.9, 1453.2, 1243.8, 1175.1 cm⁻¹; HRMS calcd. 427.2517, found 427.2511.

EXAMPLE 2—Formation of 1-Benzyl-2-phenyl[(4,dimethylaminoethoxy) phenyl]but-1-ene (Generic Type II)

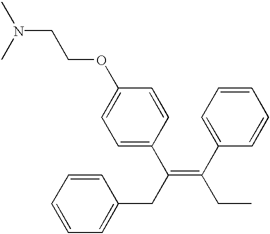

1-Benzyl-2-phenyl[(4-dimethyle aminoethoxy) phenyl]but-1-ene was prepared from 2-Phenyl-(4-dimethylaminoethoxyphenyl)ethan-1-one and propiophenone as described in example 1 above. The pure product was isolated in 49% yield following flash chromatography (CH₂Cl₂/ MeOH (40:60)—(product homogenous on TLC with rf=0.41; 60/40 MeOH/CH₂Cl₂)). HPLC RT=12.6, 15.6 mins.¹ H-NMR (CDCl₃, 400 MHz) δ=1.01 (m, 3 H, CH₃), 2.31 (s, 6 H, N(CH₃)₂), 2.39 (m, 2 H, CH₂), 2.64 (m, 2 H, CH₂N), 2.70 (m, 2 H, CH₂O), 3.95 and 3.97 (2×s, 2 H, CH₂), 6.55 (d, 2 H, J=8.52, Ar), 6.81-7.45 (m, 12 H, Ar) ppm. ¹³C-NMR (CDCl₃, 76.7 MHz) δ=12.81 (CH₃), 27.51 (CH₃), 39.55 (CH₂), 45.25 (N(CH₃)₂), 57.72 (NCH₂), 65.07 (OCH₂), 113.02-156.05 (23×Ar C) ppm. IR (film) v ×3057.2, 2871.2, 1605.2 (C=C), 1574.2, 1508.8, 1493.1, 1453.3, 1372.6, 1242.0, 1176.8 cm⁻¹; HRMS calcd. 385.2405, found 385.2506.

Further compounds of this generic type given below were prepared by analogous methods. Further details are given in Appendix 1.

1-Benzyl-2-phenyl-[1-(4-diethylaminoethoxy)phenyl]-but-1-ene

The pure product was isolated in 41% yield following flash chromatography (CH₂Cl₂/EtOAc (90:10)—(product homogenous on TLC with rf=0.27; 60/40 MeOH/CH₂Cl₂)). HPLC RT=12.0, 15.0 mins.¹ H-NMR (CDCl₃, 400 MHz) δ=0.99 (m, 3 H, CH₃), 1.21 (m, 6 H, N(CH₃)₂), 1.73 (m, 2 H, CH₂), 2.10 (m, 4 H, N(CH₂)₂), 2.84 (m, 2 H, CH₂N), 3.66, (m, 2 H, CH₂O), 4.23 and 4.25 (2×s, 2 H, CH2), 6.90-6.94 (d, 2 H, J=8.78, Ar), 7.08-7.62 (m, 10 H, Ar), 7.91-8.08 (d, 2 H, J=8.56, Ar) ppm. ¹³C-NMR (CDCl₃, 76.7 MHz) δ=10.78 (CH₃), 27.56 (CH₂), 45.17 (CH₂), 47.47 (N(CH₂)₂), 51.25 (CH₂N), 65.49 (OCH₂), 113.86-139.93 (Ar C) ppm., IR (film) v=3058.9, 2874.2, 1600.9 (C=C), 1575.5, 1510.5, 1494.8, 1453.7, 1378.3, 1249.0, 1170.5 cm⁻¹; HRMS calcd. 413.2725, found 413.2719.

1-Benzyl-2-phenyl-[1-(4-pyrrolidinylethoxy)phenyl]-but-1-ene

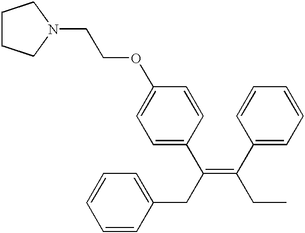

The pure product was isolated in 74% yield following flash chromatography (CH₂Cl₂/MeOH (90:10)—(product homogenous on TLC with rf=0.35; 60/40 MeOH/CH₂Cl₂)). HPLC RT=15.6, 19.2 mins.¹ H-NMR (CDCl₃, 400 MHz) δ=0.93 (t, 3 H, J=7.52, CH₃), 1.83 (m, 4 H, ((CH₂)—(CH₂)), 2.08 (q, 2 H J=7.54, CH₂), 2.67 (m, 4 H (CH₂)—N—(CH₂)), 3.02 (m, 2 H, NCH₂), 4.11 (m, 2 H, CH₂O), 4.25 (s, 2 H CH₂), 6.53-6.55 (d, 2 H, J=8.56, Ar), 6.99-7.25 (m, 12 H, Ar), 8.01-8.03 (d, 2 H, J=8.84, Ar) ppm. ¹³C-NMR (CDCl₃, 76.7 MHz)δ=8.01 (CH₃), 23.96 (CH₂), 28.25 (CH₂), 28.42 (CH₂), 55.36 (CH₂), 66.24 (OCH₂), 11426-144.09 (Ar C) ppm. IR (film) v=3058.3, 2875.0, 2225.8, 1601.9 (C=C), 1576.3, 1509.8, 1494.2, 1453.6, 1375.3, 1245.1, 1176.8 cm⁻¹; HRMS calcd. 411.2573, found 411.2562.

1-Benzyl-2-phenyl-[1-(4-pipyridinylethoxy)phenyl]-but-1-ene

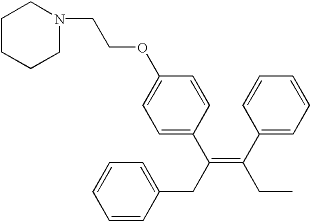

The pure product was isolated in 80% yield following flash chromatography (CH₂Cl₂/MeOH (90:10)—(product homogenous on TLC with rf=0.48; 60/40 MeOH/CH₂Cl₂)). HPLC RT=12.6, 15.0 mins.¹ H-NMR (CDCl₃, 400 MHz) δ=0.95 (m, 3 H, CH₃), 1.48 (m, 6 H, ((CH₂)-(CH₂)—(CH₂))), 2.05 (m, 2 H, CH₂), 2.70 (m, 4 H, (CH₂)—N—(CH₂)), 2.78 (m, 2 H NCH₂), 4.13 (m, 2 H, OCH₂), 4.20 (s, 2 H, CH₂), 6.49-6.51 (d, 2 H, J=8.52, Ar), 6.89-7.49 (m, 10 H, Ar), 7.91-7.98 (d, 2 H, J=8.56, Ar) ppm. ¹³C-NMR (CDCl₃, 76.7 MHz) δ=7.83 (CH₃), 24.40 (CH₂), 25.89 (CH₂), 26.10 (CH₂), 27.67 (CH₂), 45.63 (CH₂), 55.35 (CH₂), 58.09 (N(CH₂), 65.67 (OCH₂), 113.89-130.19 (Ar C) ppm. IR (film) v=3058.2, 2852.5, 1600.4 (C=C), 1575.3, 1509.3, 1494.0, 1452.7, 1372.8, 1245.1, 1175.5 cm ⁻¹; HRMS calcd. 425.2718,found 425.2719.

1-Benzyl-2-phenyl[1-(5-morpholinylethoxy)phenyl]but-1-ene

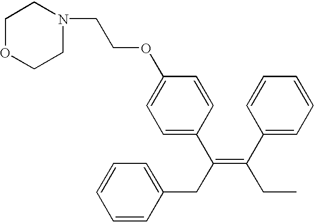

The pure product was isolated in 40% yield following flash chromatography (CH₂Cl₂/MeOH (90:10)—(product homogenous on TLC with rf=0.72; 60/40 MeOH/CH₂Cl₂)). HPLC RT=15.0, 16.2 mins.¹ H-NMR (CDCl₃, 400 MHz) δ=0.98 (t, 3 H, J=7.52, CH₃), 1.73 (q, 2 H, J=7.54, CH₂), 2.84 (m, 2 H, NCH₂), 3.02 (m, 4 H, (CH₂)—N—(CH₂)), 3.61 (m, 2 H, OCH₂), 4.12 (m, 4 H, (CH₂)—O—(CH₂)), 4.25 (s, 2 H, CH₂), 6.83-6.86 (d, 2 H, J=8.52, Ar), 6.93-7.58 (m, 10 H, Ar), 7.97-7.98 (d, 2 H, J=8.52, Ar) ppm. ¹³C-NMR (CDCl₃, 76.7 MHz) δ=7.08 (CH₃), 27.23 (CH₂), 44.24 (CH₂), 53.11 ((CH₂)—N), 53.62 (N—(CH₂)), 57.25 (CH₂N), 65.61 (OCH₂), 66.43 (2×CH₂), 113.89-130.48 (Ar C) ppm. IR (film) v=3059.0, 2856.4, 1600.5 (C=C), 1510.1, 1493.3, 1453.3, 1358.2, 1247.0, 1175.3 cm⁻¹; HRMS calcd. 427.2517, found 427.2511.

EXAMPLE 3—Formation 1-Benzyl-1-phenyl-2-[(4-dimethylaminoethoxy) phenyl]but-1-ene (Generic Type III).

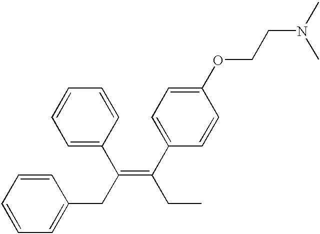

The target compound was prepared from p-dimethylaminoethoxypropiophenone and desoxybenzoin as described in example 1 above. The pure product was isolated in 24% yield following flash chromatography (CH₂Cl₂/MeOH (90:10)—(product homogenous on TLC with rf=0.13; 50/10/40 CH₂Cl₂/MeOH/EtOAc)). HPLC RT=12.0, 15.0 mins.¹ H-NMR (CDCl₃, 400 MHz)δ=0.99 (t, 3 H, J=7.54, CH₃), 2.45 (s, 6 H, (CH₃)₂), 2.63 (q, 2 H, J=7.52, CH₂), 2.86 (m, 2 H, NCH2), 3.96 (s, 2 H, CH₂), 4.07 (m, 2 H, OCH₂), 6.65-6.67 (d, 2 H, J=8.52, Ar), 6.94-7.56 (m, 10 H, Ar), 8.07-8.07 (d, 2 H, J=9.04, Ar) ppm. ¹³C-NMR (CDCl₃, 76.7 MHz) δ=12.52 (CH₃), 27.47 (CH₂), 39.59 (CH₂), 44.65 (N(CH₃)₂), 57.20 (CH₂N), 64.61 (OCH₂), 113.89-156.03 (Ar C) ppm. IR (film) v=3083.3, 2871.1, 1604.5 (C=C), 1508.6, 1494.3, 1452.8, 1381.1, 1282.4, 1177.3 cm⁻¹; HRMS calcd. 385.2405, found 385.2406.

Further compounds of this generic type given below were prepared by analogous methods. Further details are given in Appendix 1.

1-Benzyl-l-phenyl-2-[(4-diethylaminoethoxy)phenyl]but-1-ene

The pure product was isolated in 34% yield following flash chromatography (CH₂Cl₂/MeOH (80:20)—(product homogenous on TLC with rf=0.11; 50/10/40 CH₂Cl₂/MeOH/EtOAc)). HPLC RT=15.0, 19.2 mins. 1 H-NMR (CDCl₃, 400 MHz) δ=1.01 (t, 3 H, J=7.54, CH₃), 1.14 (m, 6 H, (CH₃)₂), 2.68 (m, 6 H, CH₂, N(CH₂)₂), 2.90 (t, 2 H, J=6.26, CH₂N), 3.98 (s, 2 H, CH₂), 4.03 (t, 2 H, J=6.04, OCH₂), 6.66-6.68 (d, 2 H, J=8.52. Ar), 6.94-7.31 (m, 12 H, Ar) ppm. ¹³C-NMR (CDCl₃, 76.7 MHz) δ=11.06 (CH₃), 12.54 (CH₃), 27.49 (CH₂), 39.16 (CH₂), 47.31 (N(CH₂)₂), 51.12 (CH₂N), 65.48 (OCH₂), 113.89-156.26 (Ar C) ppm. IR (film) v=3080.2, 2931.2, 1605.6 (C=C), 1508.7, 1493.6, 1453.0, 1372.6, 1283.2, 1176.4 cm⁻¹; HRMS calcd. 413.2725, found 413.2719.

1-Benzyl-1-phenyl-2-[(4-pyrrolidinylethoxy)phenyl]but-1-ene

The pure product was isolated in 24% yield following flash chromatography (CH₂Cl₂/MeOH (85:15)—(product homogenous on TLC with rf=0.30; 50/10/40 CH₂Cl₂/MeOH/EtOAc)). HPLC RT=12.6, 17.6 mins.¹ H-NMR (CDCl₃, 400 MHz) δ=0.99 (t, 3 H, J=7.00, CH₃), 1.95 (m, 4 H, (CH₂)₂), 2.65 (q, 2 H, J=7.01, CH₂), 2.98 (m, 4 H, N(CH₂)₂), 3.14 (t, 2 H, J=5.04, NCH₂), 3.96 (s, 2 H, CH₂), 4.17 (t, 2 H, J=5.26, OCH₂), 6.64-6.66 (d, 2 H, J=8.52, Ar), 6.91-7.58 (m, 10 H, Ar), 8.03-8.05 (d, 2 H, J=8.52, Ar) ppm. ¹³C-NMR (CDCl₃, 76.7 MHz) δ=12.50 (CH₃), 22.86 (CH₂), 22.92 (CH₂), 27.46 (CH₂), 39.59 (CH₂), 45.05 (CH₂N), 53.81, 53.93 (N(CH₂)₂), 64.75 (OCH₂), 113.58-155.69 (Ar C) ppm. IR (film) v=3059.8, 2928.2, 1603.2 (C=C), 1509.1, 1494.6, 1451.0, 1377.5, 1277.7, 1177.5 cm⁻¹; HRMS calcd. 411.2573, found 411.2562.

1-Benzyl-1-phenyl-2-[(4-pipyridinylethoxy)phenyl]but-1-ene

The pure product was isolated in 39%, yield following flash chromatography (CH₂Cl₂/MeOH (90:10)—(product homogenous on TLC with rf=0.40; 50/10/40 CH₂Cl₂/MeOH/EtOAc)). HPLC RT=13.2, 16.2 mins.¹ H-NMR (CDCl₃, 400 MHz) δ=0.96 (t, 3 H, J=7.52, CH₃), 1.51 (m, 2 H, CH₂), 1.75 (m, 4 H, (CH₂)₂), 2.66 (q, 2 H, J=7.54, CH₂), 2.75 (m, 4 H, N(CH₂)₂), 2.98 (m, 2 H, NCH₂), 3.95 (s, 2 H, CH₂), 4.14 (m, 2 H, OCH₂), 6.63-6.65 (d, 2 H, J=8.56, Ar), 6.75-7.56 (m, 10 H, Ar), 8.02-8.10 (d, 2 H, J=8.64, Ar) ppm. ¹³C-NMR (CDCl₃, 76.7 MHz) δ=12.90 (CH₃), 23.40 (CH₂), 24.72 (CH₂), 27.86 (CH₂), 39.99 (CH₂), 54.39 (N(CH₂)₂), 57.13 (CH₂N), 64.64 (OCH₂), 113.59-156.19 (Ar C) ppm. IR (film) v=3060.0, 2856.9, 1604.9 (C=C), 1509.1, 1494.5, 1452.9, 1379.9, 1281.5, 1177.7 cm. ¹; HRMS calcd. 425.2718, found 425.2719.

1-Benzyl-1-phenyl-2-[(4-morpholinylethoxy)phenyl]but-1-ene

The pure product was isolated in 29% yield following flash chromatography (CH₂Cl₂/MeOH (90:10)—(product homogenous on TLC with rf=0.58; 50/10/40 CH₂Cl₂/MeOH/EtOAc)). HPLC RT=12.6, 15.6 mins.¹ H-NMR (CDCl₃, 400 MHz) δ=0.99 (t, 3 H, J=7.54, CH₃), 2.63 (m, 4 H, N(CH₂)₂), 2.83 (t, 2 H, J=5.52, NCH₂), 2.89 (q, 2 H, J=7.03, CH₂), 3.75 (m, 4 H, O(CH₂)₂), 3.96 (s, 2 H, CH₂), 4.06 (t, 2 H, J=5.52, OCH₂), 6.65-6.67 (d, 2 H, J=8.56, Ar), 6.92-7.56 (m, 12 H, Ar) ppm. ¹³C-NMR (CDCl₃, 76.7 MHz), 6=12.52 (CH₃), 27.48 (CH₂), 39.61 (CH₂), 55.44 (CH₂), 57.09 (NCH₂), 64.92 (OCH₂), 66.22 ((CH₂)₂O), 113.22-156.18 (Ar C) ppm. IR (film) v=3059.4, 2929.2, 1605.8 (C=C), 1509.1, 1494.4, 1453.3, 1370.4, 1282.0, 1177.3 cm⁻¹; HRMS calcd. 427.2517, found 427.2511.

EXAMPLE 4—Formation of 1,2-Diphenyl-2-[2-(dimethylaminoethoxy) benzyl]but-1-ene (Generic Type IV). 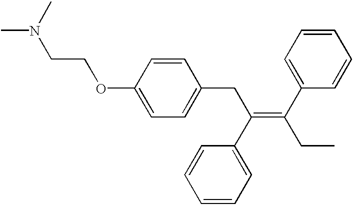 1,2-Diphenyl-2-[2-(dimethylaminoethoxy) benzyl]but-1-ene was prepared from 2-(4-Dimethylaminoethoxyphenyl)-1-phenylethanone and propiophenone as described in example 1 above. The pure product was isolated in 21% yield following flash chromatography (CH₂Cl₂/ MeOH (95:5)—(product homogenous on TLC with rf=0.50; 50/50 CH₂Cl₂/MeOH)). HPLC RT=13.2, 15.6 mins.¹ H-NMR (CDCl₃, 400 MHz) δ=1.46 (t, 3 H, J=6.86, CH₃), 2.07 (s, 6 H, (CH₃)₂), 2.74 (m, 2 H, NCH₂), 3.76 (q, 2 H, J=6.85, CH₂), 3.94 (s, 2 H, CH₂), 4.14 (m, 2 H, OCH₂), 6.52-6.56 (m, 2 H, Ar), 6.83-7.54 (m, 12 H, Ar) ppm. ¹³C-NMR (CDCl³, 76.7 MHz) δ=14.19 (CH₃), 29.68 (CH₂), 38.67 (CH₂), 47.43, 47.54 (N(CH₃)₂), 58.48 (CH₂N), 60.38 (OCH₂), 113.89-164.59 (Ar C) ppm. IR (film) v=3120.4, 2885.6, 1603.7 (C=C), 1508.7, 1496.1, 1450.5, 1382.3, 1282.4 cm⁻¹; HRMS calcd. 385.2405, found 385.2406.

Further compounds of this generic type given below were prepared by analogous methods. Further details are given in Appendix 1.

1,2-Diphenyl-2-[2-(diethylaminoethoxy)benzyl]but-1-ene

The pure product was isolated in 40% yield following flash chromatography (CH₂Cl₂/MeOH (96:4)—(product homogenous on TLC with rf=0.33; 50/50 CH₂Cl₂/MeOH)). HPLC RT=13.2, 15.0 mins. ¹ H-NMR (CDCl₃, 400 MHz) δ=0.86 (m 6 H (CH₃)₂), 0.98 (t, 3 H, J=7.52, CH₃), 2.36 (s, 4 H, (CH₂)2), 2.66 (q, 2 H, J=7.52, CH₂), 2.76 (m, 2 H, NCH₂), 3.92 (s, 2 H, CH₂), 4.08 (m, 2 H, OCH₂), 6.54-6.56 (d, 2 H, J=9.00, Ar), 6.80-7.35 (m, 12 H, Ar) ppm. IR (film) v=3059.2, 2850.6, 1604.9 (C=C), 1509.9, 1494.2, 1454.2, 1373.2, 1283.2, 1172.4 cm⁻¹; HRMS calcd. 413.2725, found 413.2719.

1,2-Diphenyl-2-[(4-pyrrolidinylethoxy)benzyl]but-1-ene

The pure product was isolated in 19% yield following prep thin layer chromatography (CH₂Cl₂/EtOAc/MeOH (50:40:10)—(product homogenous on TLC with rf=0.65; 50/50 CH₂Cl₂/MeOH)). HPLC RT=12.6, 14.4 mins.¹ H-NMR (CDCl₃, 400 MHz) δ=0.90 (t, 3 H, J=7.28, CH₃), 2.63 (m, 4 H, (CH₂)₂), 2.28 (q, 2 H, J=7.04, CH₂), 3.65 (m, 4 H, (CH₂)₂), 3.78 (m, 2 H, NCH₂), 4.15 and 4.16 (2×s, 2 H, CH₂), 4.35 (m, 2 H, OCH₂), 7.25-7.47 (m, 14 H, Ar) ppm. ¹³C-NMR (CDCl₃, 76.7 MHz) δ=17.58 (CH₃), 24.17 (CH₂), 29.21 (CH₂), 39.50 (CH₂), 47.07 (NCH₂), 63.28 (N(CH₂)₂), 66.36 (OCH₂), 116.39-146.26 (Ar C) ppm. IR (film) v=3104.7, 2973.9, 1600.4 (C=C), 1492.4, 1448.6, 1370.4, 1246.1, 1166.0cm. ¹; HRMS calcd. 411.2573, found 411.2562.

1,2-Diphenyl-2-[(4-pipyridinylethoxy)benzyl]but-1-ene

The pure product was isolated in 38% yield following flash chromatography (CH₂Cl₂/MeOH (97:3)—(product homogenous on TLC with rf=0.55; 50/50 CH₂Cl₂/MeOH)). )). HPLC RT=12.0, 15.0 mins. ¹ H-NMR (CDCl₃, 400 MHz) δ=1.01 (t, 3 H, J=7.54, CH₃), 2.07 (m, 2 H, CH₂), 2.31 (m, 4 H, (CH₂)₂), 2.58 (t, 2 H, J=7.52, CH₂), 2.74 (m, 4 H, (CH₂)₂), 3.03 (m, 2 H, NCH₂), 3.63 (s, 2 H, CH₂), 4.30 (m, 2 H, OCH₂), 6.79-7.18 (m, 12 H, Ar), 7.92-8.00 (d, 2 H, J=9.04, Ar) ppm. ¹³C-NMR (CDCl₃, 76.7 MHz) δ=13.61 (CH₃), 23.11 (CH₂), 26.81 (CH₂), 27.22 (CH₃), 31.45 (CH₂), 40.91 (CH₂), 53.66 (N(CH₂)₂), 56.08 (CH₂N), 61.07 (OCH₂), 115.09-142.76 (Ar C) ppm. IR (film) v=3060.7, 2927.1, 1601.2 (C=C), 1511.1, 1494.5, 1451.1, 1377.6, 1247.2 cm⁻¹; HRMS calcd. 425.2718, found 425.2719.

1,2-Diphenyl-2-[(4-morpholinylethoxy)benzyl]but-1-ene

The pure product was isolated in 29% yield following prep thin layer chromatography (Petroleum ether (40-60)/EtOAc (90:10)—(product homogenous on TLC with rf=0.40; 80/20 EtOAc/Pet. ether)). HPLC RT=13.2, 16.2 mins. ¹ H-NMR (CDCl₃, 400 MHz) δ=0.99 (t, 3 H, J=7.52, CH₃), 2.27 (m, 4 H, (CH₂)₂), 2.68 (q, 2 H, J=7.48, CH₂), 2.74 (t, 2 H, J=6.26, NCH₂), 3.89 (m, 4 H, CH₂), 4.64 (t, 2 H, J=6.26, OCH₂), 4.72 (s, 2 H, CH₂), 6.46-6.48 (d, 2 H, J=8.52, Ar), 6.66-7.32 (m, 10 H, Ar), 7.38-7.39 (d, 2 H, J=8.52, Ar) ppm. ¹³C-NMR (CDCl₃, 76.7 MHz) 6=13.34 (CH₃), 29.63 (CH₂), 39.89 (CH₂), 41.45 (NCH₂), 55.45 (O(CH₂)₂), 63.71 (CH₂)₂N), 64.17 (OCH₂), 114.29-128.86 (Ar C) ppm. IR (film) v=3083.0, 2973.7, 1600.4 (C=C), 1509.8, 1492.8, 1462.8, 1370.5, 1256.7, 1142.9 cm ¹; HRMS calcd. 427.2517, found 427.2511.

EXAMPLE 5—Formation of 2-[2′-methoxybenzyl]-1-[4′-(trimethylacetoxy phenyl)]-1-[4-(Pyrollidinylethoxyphenyl)]but-1-ene (Generic Type V).

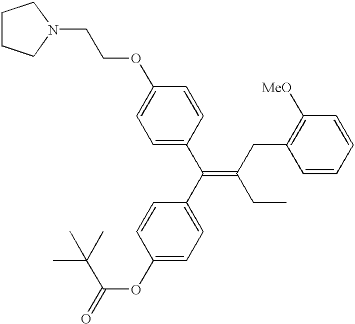

TiCl₄ (28 mmol) was added slowly to a stirring suspension of zinc powder (56 mmol) in dry THF in an inert atmosphere. This mixture was stirred for 30 min at room temperature, before being brought to reflux temperature for a further 2 hours. After this time a 1:1 mixture (7 mmol) of 4,4′-trimethylacetoxy hydroxy benzophenone and 2-methoxy phenylbutanone was added. Reflux was maintained for an additional 5 hours before quenching the reaction by pouring onto crushed ice. This solution was washed with 10% K₂CO₃ and extracted liberally with ethyl acetate to yield 2-[2′-methoxybenzyl]-1-[4′-(trimethylacetoxyphenyl)]-1-[4-(hydroxyphenyl)]but-1-ene in 36% yield following chromatography (CH₂Cl₂/EtOAc (19:1)). This compound was placed in a 100 ml two-necked round bottomed flask equipped with a magnetic stirrer and dissolved in 30 ml dry acetone. To this solution, anhydrous K₂CO₃ (3.5 g, 2.5 mmol) was added with continual stirring. Stirring was maintained for 15 minutes. After this time 1-(2-cholorethyl)pyrrolidine hydrochloride (0.8 g, 5.75 mmol) was added. Stirring was continued for a further ten minutes after which time the mixture was heated to reflux temperature for 6 h. The reaction mixture was vacuum filtered, and the residue washed with cold dry acetone. The filtrate was concentrated using reduced pressure rotary evaporation to yield a brown oil. The crude product was purified by column chromatography (silica gel) with CH₂Cl₂/MeOH 50:50 to yield the pure product (28%) as an orange-brown oil.

Further compounds of this generic type as shown below were prepared by analogous methods. Details are given in Appendix 1.

2,2-Dimethyl-propionic acid 4-{2-(2-methoxybenzyl)-1-[4-(2-pyrrolidin-1-yl-ethoxy)-phenyl]-but-1-enyl}-phenyl ester

NMR data δ(CDCl₃) 7.25-6.62(12 H, m, aromatic H), 4.12(2 H, m, CH₂), 3.80(3 H,s, CH₃), 3.56(2 H, m, CH₂), 2.97(2 H, m, CH₂), 2.73 (4 H, m, CH₂), 2.06(2 H, m, CH₂), 1.81(4 H, m, CH₂), 1.36(9 H, s, C(CH₃)₃), 0.96(3 H, s, CH₃).

4-{2-Benzyl-1-[4-(2-dimethylamino-ethoxy)-phenyl]-but-1-enyl}-phenol

NMR data δ(CDCl₃) 7.05-6.68(13 H, m, aromatic H), 6.00, (1 H, s, broad, OH), 4.10(2 H, m, CH₂), 3.57(2 H, m, CH₂), 2.93(2 H, m, CH₂), 2.51(6 H, s, N(CH₃)₂), 2.08, (2 H, m, CH₂), 0.96(3 H, m, CH₃)

2,2-Dimethyl-propionic acid 4-{2-benzyl-1-[4-(2-pyrrolidin-1-yl-ethoxy-phenyl]-but-1-enyl}-phenyl ester

NMR data δ(CDCl₃) 6.85-7.30(13 H, m, aromatic H), 4.20(2 H, m, CH₂), 3.55(2 H, m, CH₂), 3.14(2 H, m, CH₂), 2.62(4 H, m, CH₂), 2.05(2 H, m, CH₂), 1.78(4 H, m, CH₂), 1.33(9 H, m, C(CH₃)₃), 0.97(3 H, m, CH₃)

2,2-Dimethyl-propionic acid 4-{2-benzyl-1-[4-(2-pyrrolidin-1-yl-ethoxy-phenyl]-but-1-enyl}-phenyl ester

NMR data δ(CDCl₃) 6.78-7.46(13 H, m, aromatic H), 4.16(2 H, m, CH₂), 3.58(2 H, m, CH₂), 3.00(2 H, m, CH₂), 2.79(4 H, m, CH₂), 2.07(2 H, m, CH₂),1.92(4 H, m, CH₂),1.47(9 H, m, C(CH₃)₃),0.99(3 H, m, CH₃)

EXAMPLE 6—Formation of 1-(2-{14-[2-(4-methoxy-3-methyl-benzyl)-1-(4-methoxy-3-methyl-phenyl)-but-1-enyl]-phenoxy}-ethyl) -pyrrolidine.

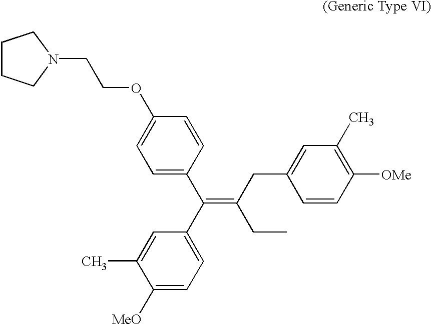

The target compound was prepared from the initial titanium mediated coupling of (4-hydroxy-phenyl)-(4-methoxy-3-methyl-phenyl)-methanone to 1-(4-methoxy-3-methyl-phenyl)-butan-2-one, followed by alkylation of the formed intermediate compound's free hydroxy group with 1,(2-chloroethyl)pyrrolidine hydrochloride and subsequent chromatographic purification as described in example 5 above.

EXAMPLE 7—Formation of 4-(2-benzyl-1-phenylbut-1-enyl)phenyl-amine (intermediate)

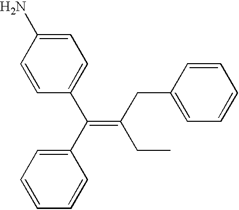

Zinc (64 mmol, 4.184 g) and titanium tetrachloride (32 mmol, 3.64 ml) were dissolved in dry tetrahydrofuran (THF) under nitrogen and brought to reflux for 2 hours. A solution of 4-aminobenzophenone (8 mmol, 1.578 g) and phenylbutanone (24 mmol, 3.557 g) in dry THF (50 ml) was added and the mixture refluxed for a further 4 hours. The reaction mixture was allowed cool to room temperature and was poured onto 5% aq. NaHCO₃ (1000 ml). This was extracted with ethyl acetate (EtOAc) (4×200 ml) and dichloromethane (DCM) (200 ml). The combined organic phases were dried over anhydrous Na₂SO₄, filtered and evaporated to dryness in vacuo. 4-(2-Benzyl-1-phenylbut-1-enyl)-phenylamine was isolated as a light brown oil in an E/Z mixture in an 89% yield following flash column chromatography on silica gel using hexane:diethyl ether 3:1 as mobile phase.

EXAMPLE 8—Formation of N-[4-(2-benzyl-1-phenylbut-1-enyl)-phenyl]-2,2,2-trifluoroacetamide (intermediate)

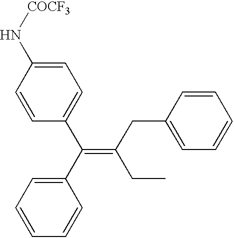

4-(2-Benzyl-1-phenyl-but-1-enyl)-phenylamine (6.24 mmol, 1.955 g) was dissolved in anhydrous DCM (50 ml) under nitrogen and was cooled to 0° C. Triethylamine (6.55 mmol, 0.94 ml) was added to the mixture, followed by the dropwise addition of trifluoroacetic anhydride (9.36 mmol, 1.34 ml). The mixture was stirred at 0° C. for a further 90 minutes and was allowed return to room temperature. This was then washed with 10% HCl (2×30 ml), dried over anhydrous Na₂SO₄, filtered and evaporated to dryness in vacuo. N-[4-(2-Benzyl-1-phenylbut-1-enyl)-phenyl]-2,2,2-trifluoroacetamide was recovered as a light brown solid that was further purified by recrystallisation from hot diethylether/hexane to yield a white crystalline solid consisting of the single Z isomer.

EXAMPLE 9—Formation of N-[4-(2-benzyl-1-phenylbut1enyl)-phenyl]-2,2,2-trifluoro-N-[3-(tetrahydropyran-2-yloxy)-propyl]-acetamide (Intermediate)

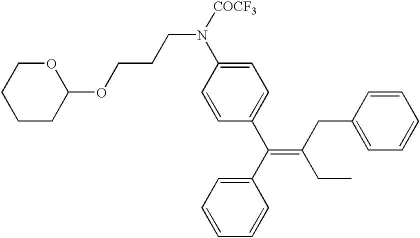

N-[4-(2-Benzyl-1-phenylbut-1-enyl)-phenyl]-2,2,2-trifluoroacetamide (2 mmol, 820 mg), was dissolved in DMF (10 ml) and anhydrous potassium carbonate (K₂CO₃) (7.6 mmol, 1.05 g) was added. This was heated to 100° C., at which point 2-(3-bromopropoxy)-tetrahydropyran (5.66 mmol, 1.27 g) was added. This was heated for a further 90 minutes and was allowed cool to room temperature. The reaction mixture was diluted with DCM (50 ml) and washed with water (6×100 ml). The organic phase was dried over anhydrous Na₂SO₄, filtered and evaporated to dryness in vacuo. N-[4-(2-Benzyl-1-phenylbut-1-enyl)-phenyl]-2,2,2-trifluoro-N-[3-(tetrahydropyran-2-yloxy)-propyl]-acetamide was isolated as a clear colorless oil in a 93% yield following flash column chromatography on silica gel using hexane:diethyl ether 3:1 as mobile phase.

EXAMPLE 10—Formation of [4-(2-Benzyl-1-phenylbut-1-enyl)-phenyl]-[3-(tetrahydropyran-2-yloxy)-propyl]-amine (Intermediate)

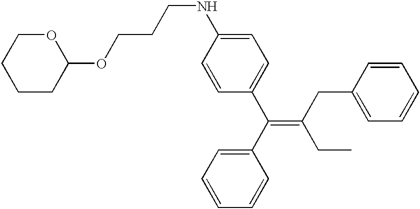

N-[4-(2-Benzyl-1-phenylbut-1-enyl)-phenyl]-2,2,2-trifluoro-N-[3-(tetrahydropyran-2-yloxy)-propyl]-acetamide (2 mmol, 1.103 g) was dissolved in a 10:1 mixture of methanol and water (22 ml) and K₂CO3 (10 mmol, 2.77 g) was added. This was refluxed for 20 minutes and was allowed cool to room temperature. Excess methanol was removed in vacuo and the residue was diluted with water (20 ml). This was extracted with DCM (2×20 ml) and the organic phase was dried over over anhydrous Na₂SO₄, filtered and evaporated to dryness in vacuo. [4-(2-Benzyl-1-phenylbut-1-enyl)-phenyl]-[3-(tetrahydropyran-2-yloxy)-propyl]-amine was recovered in a 90% yield as a clear colorless oil, without the need for further purification.

EXAMPLE 11—Formation of [4-(2-benzyl-1-phenylbut-1-enyl)-phenyl]-[3-(tetrahydropyran-2-yloxy)-propyl]-carbamic acid ethyl ester (intermediate)

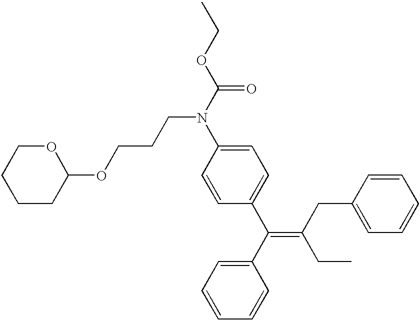

[4-(2-Benzyl1-phenylbut1enyl)-phenyl]-[3-(tetrahydropyran-2-yloxy)-propyl]-amine (1.7 mmol, 768 mg) was dissolved in dry DCM (20 ml) under nitrogen and cooled to 0° C. Triethylamine (2.53 mmol, 355 μl) was added to the mixture, which was stirred for a further 5 minutes. Ethylchloroformate (2.53 mmol, 241 μl ) was added dropwise to the mixture which was then stirred overnight at room temperature. The reaction mixture was washed with 5% HCl (2×25 ml), dried over over anhydrous Na₂SO₄, filtered and evaporated to dryness in vacuo. [4-(2-Benzyl-1-phenylbut-1 -enyl)-phenyl]-[3-(tetrahydropyran-2-yloxy)-propyl]-carbamic acid ethyl ester was isolated as a clear oil in an 86% yield following flash column chromatography on silica gel using hexane:diethyl ether 3:1 as mobile phase.

EXAMPLE 12—Formation of [4-(2-benzyl-1-phenylbut-1-enyl)-phenyl]-(3-hydroxypropyl)-carbamic acid ethyl ester (Intermediate)

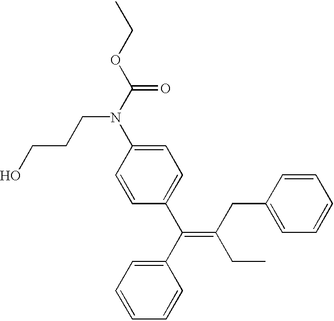

[4-(2-Benzyl 1-phenylbut-1-enyl)-phenyl]-[3-(tetrahydropyran-2-yloxy)-propyl]-carbamic acid ethyl ester (1.25 mmol, 661 mg) was dissolved in methanol and p-toluene sulphonic acid (1.5 mmol, 283 mg) was added. The mixture was stirred at room temperature for 30 minutes and excess methanol was removed in vacuo. The residue was taken up in DCM (20 ml) and washed with water (20 ml), dried over over anhydrous Na₂SO₄, filtered and evaporated to dryness in vacuo. [4-(2-Benzyl-1-phenylbut-1-enyl)-phenyl]-(3-hydroxypropyl)-carbamic acid ethyl ester was isolated as a light yellow oil in 98% yield following flash column chromatography on silica gel using hexane:diethyl ether 1:1 as mobile phase.

EXAMPLE 13—Formation of [4-(2-benzyl-1-phenylbut-1-enyl)-phenyl]-(3-ethoxypropyl)-amine (Generic Type VIII)

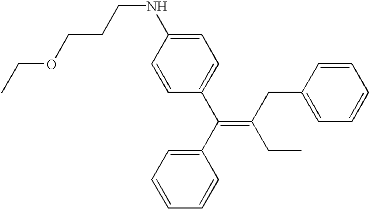

[4-(2-Benzyl-1 -phenylbut-1 -enyl)-phenyl]-(3-hydroxypropyl)-carbamic acid ethyl ester (0.33 mmol, 145 mg) was dissolved in dry THF under nitrogen and iodoethane (3.3 mmol, 240 μl) was added to the mixture. Sodium hydride (60% dispersion in mineral oil) (0.66 mmol, 26 mg) was carefully added to the mixture which was then stirred overnight at room temperature. The reaction was quenched by the dropwise addition of methanol (1 ml) followed by the dropwise addition of water (1 ml) to the mixture. Excess THF was removed in vacuo, and the residue taken up in DCM (20 ml). This was washed with saturated aqueous NH₄Cl (2×20 ml), dried over anhydrous Na₂SO_{4,} filtered and evaporated to dryness in vacuo. This was further purified by flash column chromatography on silica gel using hexane:diethyl ether 4:1 as mobile phase to yield [4-(2-benzyl-1-phenylbut-1-enyl)-phenyl]-(3-ethoxypropyl)-amine as a colorless opaque oil in a 63% yield.

IRvₘₐₓ (film) 3393 (NH), 2870 (CH) cm⁻¹. ¹ H NMR δ (CDCl₃) 1.00 (3 H, t, J=7.3 Hz, H-4), 1.26 (3 H, t, J=7.0 OHz, OCH₂CH₃), 1.91 (2 H, m, OCH₂CH₂CH₂N), 2.08 (2 H, q, J=7.4 Hz, H-3), 3.24 (2 H, t, J=6.5 Hz, NCH₂), 3.52 (2 H, q, J=7.0 Hz, OCH₂CH₃), 3.58 (2 H, t, J=5.8 Hz, OCH₂CH₂CH₂N), 3.58 (2 H,s, CCH₂Ar), 4.04 (1 H, bs, NH), 6.56 (2 H, d, J=8.6 Hz, H-3′,5′), 7.09 (2 H, d, J=8.5 Hz, H-2′,6′), 7.22-7.36 (10 H, m, Ar—H).¹³C NMR ppm (CDCl₃) 13.29 (C-4), 15.19 (OCH₂CH₃), 24.74 (C-3), 29.42 (NCH₂CH₂CH₂₀), 37.18 (CCH₂Ar), 42.05 (NCH₂), 66.27 (OCH₂CH₃), 69.12 (OCH₂CH₂CH₂N), 112.26 (C-3′,5′), 125.63, 125.92 (C-4″,4′″), 127.88, 128.19, 128.70, 129.28 (H-2″,6″,2′″6,′″), 130.13 (C-2′,6′), 131.96 (C-1′), 137.56, 139.51, 140.99, 143.90 (C-1,2,1″,1′″), 146.89 (C-4′). m/z 400 (^{M+}+1, 100%). HRMS calcd. for C₂₈ H₃₄NO (M⁺+H) 400.2640. Found 400.2647

Further compounds of this generic type given below were prepared by analogous methods. Further details are given in Appendix 2.

[4-(2-Benzyl1-phenylbut1enyl)-phenyl]-(2-methoxyethyl)-amine

IRvₘₐₓ(film) 3394 (NH) cm⁻¹. ¹ H NMR δ (CDCl₃) 1.00 (3 H, t, J=7.5 Hz, H-4), 2.08 (2 H, q, J=7.5 Hz, H-3), 3.29 (2 H, t, J=5.3 Hz, NCH₂), 3.41 (3 H, s, OCH₃), 3.62 (2 H, t, J=5.3 Hz, OCH₂), 3.67 (CCH₂Ar), 3.56 (2 H, t, J=5. OHz, OCH₂), 6.59 (2 H, d, J=8.9 Hz, H-3′,5′), 7.09 (2 H, d, J=9. OHz, H-2′,6′), 7.22-7.36 (10 H, m, Ar—H). ¹³C NMR ppm (CDCl₃) 13.29 (C-4), 24.75 (C-3), 37.18 (CCH₂Ar), 43.43 (NCH₂), 58.64 (OCH₃), 71.03 (OCH₂), 112.62 (C-3′,5′), 125.66, 126.96 (C-4″,4′″), 127.91, 128.21, 128.70, 129.28 (H-2″,6″,2′″,6′″), 130.15 (C-2′,6′), 132.45 (C-1′), 137.69, 139.44, 140.96, 143.86 (C-1,2,1″,1′″), 146.54 (C-4′). m/z 372 (M⁺+1, 100%). HRMS calcd. for C₂₆ H₃₀NO (M⁺+H) 372.2327. Found 372.2330.

[4-(2-Benzyl-1-phenylbut-1-enyl)-phenyl]-(2-ethoxyethyl)-amine

IRvₘₐₓ (film) 3403 (NH), 2927 (CH) cm-⁻¹. ¹ H NMR δ (CDCl₃) 0.98 (3 H, t, J=7.5 Hz, H-4), 1.24 (3 H, t, J=7. OHz, OCH₂CH₃), 2.06 (2 H, q, J=7.4 Hz, H-3), 3.28 (2 H, t, J=5.3 Hz, NCH₂), 3.54 (2 H, q, J=7.0 Hz, OCH₂CH₃), 3.54 (2 H, q, OCH₂CH₃), 3.63-3.65 (4 H, m, OCH₂CH₂N, CCH₂Ar), 6.58 (2 H, d, J=8.0 Hz, H-3′,5′), 7.08 (2 H, d, J=8.5 Hz, H-2′,6′), 7.21-7.34 (10 H, m, Ar—H). ¹³C NMR ppm (CDCl₃) 13.30 (C-4), 15.12 (OCH₂CH₃), 24.77 (C-3), 37.20 (CCH₂Ar), 43.70 (NCH₂), 66.33 (OCH₂CH₃), 68.87 (OCH₂CH₂N), 112.73 (C-3′,5′), 125.67, 125.97 (C-4″,4′″), 127.92, 128.22, 128.72, 129.30 (H-2″,6″,2′″,6′″), 130.16 (C-2″,6′), 132.52 (C-1′), 137.72, 139.46, 140.98, 143.87, 146.53 (C-1,2,1″,1′″,4′). m/z 386 (M⁺+1, 100%), 313 (3). HRMS calcd. for C₂₇ H₃₂NO (M⁺+H) 386.2484. Found 386.2474.

[4-(2-Benzyl1-phenylbut1enyl)-phenyl]-(2-propoxyethyl)-amine

IRvₘₐₓ (film) 3404 (NH), 2924 (CH) cm-⁻¹. ¹ H NMR δ (CDCl₃) 0.91-0.99 (6 H, m, H-4, OCH₂CH₂CH₃), 1.58-1.72 (2 H, m, OCH₂CH₂CH₃), 2.04 (2 H, q, J=7.5 Hz, H-3), 3.29 (2 H, t, J=5.3 Hz, NCH₂), 3.42 (2 H, t, J =6.8 Hz, OCH₂CH₂CH₃), 3.62 (2 H, s, CCH₂Ar), 3.64 (2 H, t, J=5.5 Hz, OCH₂CH₂N), 6.67 (2 H, d, J=8.5 Hz, H-3′,5′), 7.08 (2 H, d, J=8.5 Hz, H-2′,6′), 7.21-7.33 (10 H, m, Ar—H). ¹³C NMR ppm (CDCl₃) 10.51 (C-4, OCH₂CH₂CH₃), 13.29 (C-4), 22.85 (OCH₂CH₂CH₃), 24.79 (C-3), 39.40 (CCH₂Ar), 44.59 (NCH₂), 68.54 (OCH₂CH₂N), 72.78 (OCH₂CH₂CH₃), 113.89 (C-3′,5′), 125.71, 126.05 (C-4″,4′″), 127.97, 128.25, 128.71, 129.30 (H-2″,6″,2′″,6′″), 130.64 (C-2′,6′), 132.97, 132.71, 138.02, 139.32, 140.88 (C-1,2,1′,1″,1′″), 143.71 (C-4′).

m/z 400 (M⁺+1, 100%), 312 (5). HRMS calcd. for C₂₈ H₃₄NO (M⁺+H) 400.2640. Found 400.2614.

[4-(2-Benzyl1-phenylbut1enyl)-phenyl]-(3-methoxypropyl)-amine

IRvₘₐₓ (film) 3400 (NH) cm-⁻¹. ¹ H NMR δ (CDCl₃) 1.00 (3 H, t, J=7.5 Hz, H-4), 1.91 (2 H, m, NCH₂CH₂CH₂O), 2.08 (2 H, q, J=7.4 Hz, H-3), 3.23 (2 H, t, J=6.5 Hz, NCH₂), 3.38 (3 H, s, OCH₃), 3.53 (2 H, t, J=5.8 Hz, OCH₂), 3.68 (2 H, s, CCH₂Ar), 6.56 (2 H, d, J=8.6 Hz, H-3′,5′), 7.09 (2 H, d, J=8.5 Hz, H-2′,6′), 7.22-7.36 (1OH, m, Ar—H). ¹³C NMR ppm (CDCl₃) 13.29 (C-4), 24.75 (C-3), 29.38 (OCH₂CH₂CH₂N), 37.19 (CCH₂Ar), 41.80 (NCH₂), 58.66 (OCH₃), 71.19 (OCH₂), 112.28 (C-3′,5′), 125.64, 125.93 (C-4″,4′″), 127.89, 128.20, 128.70, 129.28 (H-2″,6″,2′″6,′″), 130.15 (C-2′,6′), 132.02 (C-1′), 137.59, 139.50, 140.99, 143.90 (C-1,2,1″,1′″), 146.81 (C-4′).

m/z 386 (M⁺+1, 100%), 313 (9). HRMS calcd. for C₂₇ H₃₂NO (M⁺+H) 386.2484. Found 386.2511.

[4-(2-Benzyl1-phenylbut1enyl)-phenyl]-(3-ethoxypropyl)-amine

IRvₘₐₓ (film) 3393 (NH), 2870 (CH) cm-⁻¹. 1 H NMR δ (CDCl₃) 1.00 (3 H, t, J=7.3 Hz, H-4), 1.26 (3 H, t, J=7.0 Hz, OCH₂CH₃), 1.91 (2 H, m, OCH₂CH₂CH₂N), 2.08 (2 H, q, J=7.4 Hz, H-3), 3.24 (2 H, t, J=6.5 Hz, NCH₂), 3.52 (2 H, q, J=7.0 Hz, OCH₂CH₃), 3.58 (2 H, t, J=5.8 Hz, OCH₂CH₂CH₂N), 3.58 (2 H,s, CCH₂Ar), 4.04 (1 H, bs, NH), 6.56 (2 H, d, J=8.6 Hz, H-3′,5′), 7.09 (2 H, d, J=8.5 Hz, H-2′,6′), 7.22-7.36 (10 H, m, Ar-H). ¹³C NMR ppm (CDCl₃) 13.29 (C-4), 15.19 (OCH₂CH₃), 24.74 (C-3), 29.42 (NCH₂CH₂CH₂O), 37.18 (CCH₂Ar), 42.05 (NCH₂), 66.27 (OCH₂CH₃), 69.12 (OCH₂CH₂CH₂N), 112.26 (C-3′,5′), 125.63, 125.92 (C-4″,4′″), 127.88, 128.19, 128.70, 129.28 (H-2″,6″,2′″6,′″), 130.13 (C-2′,6′), 131.96 (C-1′), 137.56, 139.51, 140.99, 143.90 (C-1,2,1″,1′″), 146.89 (C-4′). m/z 400 (M⁺+1, 100%). HRMS calcd. for C₂₈ H₃₄NO (M⁺+H) 400.2640. Found 400.2647.

[4-(2-Benzyl-1-phenylbut-1-enyl)-phenyl]-(3-propoxypropyl)-amine

IRvₘₐₓ (film) 3396 (NH), 2926 (CH) cm-⁻¹. ¹ H NMR δ (CDCl₃) 0.97 (3 H, t, J=7.0 Hz, OCH₂CH₂CH₃), 0.99 (3 H, t, J=7.6 Hz, H-4), 1.59-1.66 (2 H, m, OCH₂CH₂CH₃), 1.87-1.93 (2 H, m, OCH₂CH₂CH₂N), 2.06 (2 H, q, J=7.5 Hz, H-3), 3.23 (2 H, t, J=6.5 Hz, NCH₂), 3.40 (2 H, t, J=6.5 Hz, OCH₂CH₂CH₃), 3.56 (2 H, t, J=5.8 Hz, OCH₂CH₂CH₂N), 3.66 (2 H, s, CCH₂Ar), 6.54 (2 H, d, J=8.5 Hz, H-3′,5′), 7.07 (2 H, d, J=8.5 Hz, H-2′,6′), 7.21-7.34 (10 H, m, Ar—H). ¹³C NMR ppm (CDCl₃) 10.64 (OCH₂CH₂CH₃), 13.31 (C-4), 22.96 (OCH₂CH₂CH₃), 24.77 (C-3), 29.41 (NCH₂CH₂CH₂O), 37.21 (CCH₂Ar), 42.21 (NCH₂), 69.49 (OCH₂CH₂CH₂N), 72.76 (OCH₂CH₂CH₃), 112.25 (C-3′,5′), 125.65, 125.94 (C-4″,4′″), 127.90, 128.21, 128.73, 129.31 (H-2″,6″,2′″,6′″), 130.16 (C-2′,6′), 131.95 (C-1′), 137.58, 139.55, 141.03, 143.95 (C-1,2,1″,1′″), 146.95 (C-4′). m/z 414 (M⁺+1, 100%), 313 (8). HRMS calcd. for C₂₉ H₃₆NO (M⁺+H) 414.2797. Found 414.2802.

2-[4-(2-Benzyl-1-phenylbut-1-enyl)-phenylamino]-ethanol

IRvₘₐₓ (film) 3400 (OH) cm-⁻¹. ¹ H NMR δ (CDCl₃) 0.98 (3 H, t, J=7.3 Hz, H-4), 2.05 (2 H, q, J=7.5 Hz, H-3), 2.57 (2 H, bs. NH, OH), 3.28 (2 H, t, J=5.3 Hz, NCH₂CH₂OH), 3.60 (2 H, s, CCH₂Ar), 3.81 (2 H, t, J=5.3 Hz, NCH₂CH₂OH), 6.58 (2 H, d, J=8.5 Hz, H-3′,5′), 7.07 (2 H, d, J=8.5 Hz, H-2′,6′), 7.20-7.34 (10 H, m, ArH). ¹³C NMR ppm (CDCl₃) 13.28 (C-4), 24.76 (C-3), 37.19 (CCH₂Ar), 46.17 (NCH₂CH₂OH), 61.28 (NCH₂CH₂OH), 112.86 (C-3′,5′), 125.69, 126.00 (C-4″,4′″), 127.95, 128.24, 128.70, 129.27 (C-2″,3″,5″,6″,2′″,3′″,5′″,6′″), 130.23 (C-2′,6′), 132.76 (C-1′), 137.85, 139.34, 140.90, 143.79 (C-1,2,1″,1′″), 146.42 (C-4′). m/z 358 (M⁺+1, 100%), 327 (24), 91 (35).

3-[4-(2-Benzyl-1-phenylbut-1-enyl)-phenylamino]-propan-1-ol

IRvₘₐₓ (film) 3388 (OH), 2932 (CH) cm-⁻¹. ¹ H NMR δ (CDCl₃) 0.97 (3 H, t, J=7.5 Hz, H-4), 1.87 (2 H, m, NCH₂CH₂CH₂OH), 2.05 (2 H, q, J=7.4 Hz, H-3), 2.58 (2 H, bs. NH, OH), 3.26 (2 H, t, J=6.3 Hz, NCH₂), 3.64 (2 H, s, CCH₂Ar), 3.81 (2 H, t, J=5.8 Hz, CH₂OH), 6.57 (2 H, d, J=8.5 Hz, H-3′,5′), 7.07 (2 H, d, J=8.5 Hz, H-2′6′), 7.20-7.33 (10 H, m, ArH). ¹³C NMR ppm (CDCl₃) 13.29 (C-4), 24.76 (C-3), 31.93 (NCH₂CH₂CH₂OH), 37.20 (CCH₂Ar), 42.05 (NCH₂), 61.67 (CH₂OH), 112.76 (C-3′,5′), 125.29, 125.98 (C-4″,4′″), 127.93, 128.23, 128.71, 129.29 (C-2′″,31″,5″,6″,2′″,3′″,5′″,6′″), 130.20 (C-2′,6′), 132.65 (C-1′), 137.77, 139.41, 140.94, 143.84 (C-1,2,1″,1′″), 146.59 (C-4′).

2-[4-(2-Benzyl-1-phenylbut1enyl)-phenoxy]-ethanol

IRvₘₐₓ (film) 3391 (OH), 1606, 1505 (C=C) cm-⁻¹. ¹ H NMR δ (CDCl₃) 1.00 (2 H, t, J=7.5 Hz, H-4), 1.01 (1 H, t, J=7.5 Hz, H-4), 2.09 (1.33 H, q, J=8.0 Hz, H-3), 2.10 (0.67 H, q, J=7.6 Hz, H-3), 3.58 (0.67 H, s, CCH₂Ar), 3.62 (1.33 H, s, CCH₂Ar), 3.95 (1.33 H, t, J=4.5 Hz, CH₂OH), 3.97 (0.67 H, t, J=5.0 Hz, CH₂OH), 4.06 (1.33 H, t, J=4.8 Hz, OCH₂CH₂OH), 4.10 (0.67 H, t, J=4.5 Hz, OCH₂CH₂OH), 6.86 (1.33 H, d, J=8.6 Hz, H-3′,5′), 6.90 (0.67 H, d, J=8.5 Hz, H-3′,5′), 7.17-7.36 (12 H, m, Ar—H). ¹³C NMR ppm (CDCl₃) 13.23* (C-4), 24.72* (C-3), 37.15* (CCH₂Ar), 61.41* (CH₂OH), 69.12* (OCH₂CH₂OH), 114.12*, 114.20 (C-3′,5′), 125.77*, 126.17*, 126.22 (C-4″,4″), 128.02*, 128.09, 128.26, 128.49*, 128.62*, 129.18*, 129.38, 130.35*, 130.38 (ArC-H), 136.01*, 138.58*, 138.89*, 140.60*, 143.36* (C-1,2,1′,1″,1 ′″),157.08* (C-4′). m/z 358 (M⁺, 100%), 314 (39), 91 (36).

3-[4-(2-Benzyl-1-phenylbut-1-enyl)-phenoxy]-propan-1-ol

IRvₘₐₓ (film) 3392 (OH), 1605, 1508 (C=C) cm-¹. ¹ H NMR δ (CDCl₃) 1.00 (1.8 H, t, J=7.3 Hz, H-4), 1.01 (1.2 H, t, J=7.5 Hz, H-4), 2.02-2.12 (4 H, m, OCH₂CH₂CH₂OH, H-₃), 3.58 (0.8 H, s, CCH₂Ar), 3.62 (1.2 H, s, CCH₂Ar), 3.81 (0.8 H, t, J=5.8 Hz, CH₂OH), 3.85 (1.2 H, t, J=6.8 Hz, CH₂OH), 4.10 (1.2 H, t, J=6.0 Hz, OCH₂CH₂CH₂OH), 4.13 (0.8 H, t, J=6.OHz, OCH₂CH₂CH₂OH), 6.85 (1.2 H, d, J=7.0 Hz, H-3′,5′), 6.88 (0.8 H, d, J=9 Hz, H-3′,5′), 7.17-7.33 (12 H, m, Ar—H). ¹³C NMR ppm (CDC₃) 13.21* (C-4), 24.68* (C-3), 31.94 (OCH₂CH₂CH₂OH), 37.12* (CCH₂Ar), 60.32* (CH₂OH), 65.55* (OCH₂CH₂CH₂OH), 114.00*, 114.07 (C-3′,5′), 125.73*, 126.12*, 126.17 (C-4″,4′″), 127.98*, 128.05, 128.23*, 128.59*, 129.15*, 130.27* (ArC—H), 135.71*, 138.47*, 138.93*, 140.60*, 143.38* (C-1,2,1′,1″,1′″), 157.21* (C-4′).

m/z 372 (M⁺, 100%), 314 (83), 91 (19).

{4-[2-Benzyl-1-(4-methoxyphenyl)-but-1-enyl]-phenyl}-(2-methoxyethyl)-amine

IRvₘₐₓ (film) 3401 (NH), 1610, 1504 (C=C) cm-⁻¹. ¹ H NMR δ (CDCl₃) 1.08 (2.25 H, t, J=7.3 Hz, H-4), 1.10 (0.75 H, t, J=7.3 Hz, H-4), 2.18 (1.5 H, q, J=7.5 Hz, H-3), 2.23 (0.5 H, q, J=7.5 Hz, H-3), 3.34 (1.5 H, bt, J=5.0 Hz, NCH₂), 3.37 (0.5 H, bt, J=5.0 Hz, NCH₂), 3.45 (2.25 H, s, CH₂OCH₃), 3.47 (0.75 H, s, CH₂OCH₃), 3.64 (2 H, t, J=5.5 Hz, OCH₂), 3.74 (2 H, s, CCH₂Ar), 3.83 (0.75 H, s, ArOCH₃), 3.87 (2.25 H, s, ArOCH₃), 6.64 (1.5 H, d, J=8.5 Hz, H-3′,5′), 6.68 (0.5 H, d, J=8.5 Hz, H-3′,5′), 6.91 (0.5 H, d, J=8.5 Hz, H-3″,5″), 6.95 (1.5 H, d, J=8.5 Hz, H-3″,5″), 7.14 (0.5 H, d, J=8.5 Hz, H-2′,6′), 7.15 (1.5 H, d, J=8.5 Hz, H-2′,6′), 7.26 (1.5 H, d, J=8.6 Hz, H-2″,6″), 7.26 (0.5 H, d, J=8.0 Hz, H-2″,6″), 7.28-7.42 (5 H, m, H-2′″,3′″,4′″,5′″,6′″). ¹³C NMR ppm (CDCl₃) 13.27* (C-4), 24.65* (C-3), 37.15*, 37.20 (CCH₂Ar), 43.28* (NCH₂), 54.98* (ArOCH₃), 58.53*, 58.55 (CH₂OCH₃), 70.89*, 70.92 (OCH₂), 112.37, 112.44*, 113.18*, 113.24 (C-3′,5′,3″,5″), 125.55* (C-4′″). 128.12*, 128.54, 128.59*, 130.05*, 130.14, 130.24, 130.29* (C-2′,6′,2″,6″,2′″,3′″,5′″,6′″), 132.61*, 136.15, 136.19*, 137.19*, 138.82*, 140.86* (C-1,2,1′,1″,1′″), 146.41* (C-4′), 157.71*, 157.75 (C-4′).

m/z 402 (M⁺+1, 100%), 342 (23), 251 (15).

{4-[2-(4-methoxybenzyl)-1-(4-methoxyphenyl)-but-1-enyl]-phenyl}-(2-methoxyethyl)-amine

IRvₘₐₓ (film)3398 (NH), 1611, 1505 (C=C) cm-⁻¹. ¹ H NMR δ (CDCl₃) 1.08 (3 H, t, J=7.3 Hz, H-4), 2.17 (1.5 H, q, J=7.6 Hz, H-3), 2.23 (0.5 H, q, J=7.5 Hz, H-3), 3.33 (1.5 H, t, J=5.OHz, NCH₂), 3.36 (0.5 H, b, J=5.0 Hz, NCH₂), 3.44 (2.25 H, s, CH₂OCH₃), 3.46 (0.75 H, s, CH₂OCH₃), 3.63 (0.5 H, t, J=4.5 Hz, OCH₂), 3.64 (1.5 H, t, J=5.0 Hz, OCH₂), 3.67 (2 H, s, CCH₂Ar), 3.82 (0.75 H, s, ArOCH₃), 3.86 (2.25 H, s, ArOCH₃), 3.86 (3 H, s, ArOCH₃), 4.11 (1 H, bs, NH), 6.64 (1.5 H, d, J=8.5 Hz, H-3′,5′), 6.68 (0.5 H, d, J=8.6 Hz, H-3′,5′), 6.89-6.96 (4 H, m, H-3′,5′,3′″,5′″) 7.13 (0.5 H, d, J=8.0 Hz, H-2′,6′), 7.14 (1.5 H, d, J=8.5 Hz, H-2′,6′), 7.24-7.28 (4 H, m, H-2″,6″,2′″,6′″). ¹³C NMR ppm (CDCl₃) 13.22* (C-4), 24.52* (C-3), 36.17*, 36.21 (CCH₂Ar), 43.22* (NCH₂), 54.89*, 54.96* (2×ArOCH₃), 58.44* (CH₂OCH₃), 70.82*, 70.86 (OCH₂), 112.30, 112.35*, 113.11*, 113.17 (C-3′,5′,3″,5″,3′″,5′″), 129.33, 129.38*, 130.00*, 130.08, 130.20, 130.24* (C-2′,6′,2″,6″,2′″,6′″), 132.54*, 132.65, 132.73, 132.80*, 136.12, 136.19*, 137.49*, 138.47* (C-1,2,1′,1″,1′″), 146.36* (C-4′), 157.53*, 157.65*, 157.67 (C-4″,4′″).

m/z 432 (M⁺+1, 100%), 373 (61), 121 (56).

{4-[2-(4-methoxybenzyl)-1-phenylbut-1-enyl]-phenyl}-(2-methoxyethyl)-amine

IRvₘₐₓ (film) 3337 (NH), 1611, 1509 (C=C) cm-⁻¹. ¹ H NMR δ (CDCl₃) 1.01 (3 H, t, J=7.5 Hz, H-4), 2.08 (2 H, q, J=7.4 Hz, H-3), 3.30 (2 H, t, J=5.3 Hz, NCH₂), 3.41 (3 H, s, CH₂OCH₃), 3.62 (2 H, s, CCH₂Ar), 3.62 (2 H, t, J=5.0 Hz, OCH₂), 3.84 (3 H, s, ArOCH₃), 6.59 (2 H, d, J=8.5 Hz, H-3′,5′), 6.90 (2 H, d, J=8.52 Hz, H-3′″,5′″), 7.10 (2 H, d, J=8.0 Hz, H-2′,6′), 7.20 (2 H, d, J=8.5 Hz, H-2′″,6′″), 7.22-7.36 (5 H, m, Ar—H). ¹³C NMR ppm (CDCl₃) 13.26 (C-4), 24.63 (C-3), 36.22 (CCH₂Ar), 43.40 (NCH₂), 55.15 (ArOCH₃), 58.60 (CH₂OCH₃), 70.99 (OCH₂), 112.57, 113.66 (C-3′,5′,3′″,5′″), 125.89 (C-4″), 127.86, 129.25, 129.50, 130.13 (C-2′,6′,2″,3″,5″,6″,62′″,6′″), 132.44, 132.90, 138.02, 139.09 (C-1,2,1′,1′″), 143.87, 146.48 (C-4′,1″), 157.71 (C-4′″).

m/z 401 (M⁺, 100%), 343 (38), 121 (24).

1-(2-Benzyl-1-phenylbut-1-enyl)-4-(2-methoxyethoxy)-benzene IRvₘₐₓ (film) 1606, 1507 (C=C) cm-⁻¹. ¹ H NMR δ (CDCl₃) 1.08 (2.25 H, t, J=7.5 Hz, H-4), 1.09 (0.75 H, t, J=7.3 Hz, H-4), 2.17 (1.5 H, q, J=7.4 Hz, H-3), 2.20 (0.5 H, q, J=7.5 Hz, H-4), 3.50 (2.25 H, s, OCH₃), 3.52 (0.75 H, s, OCH₃), 3.67 (0.5 H, s, CCH₂Ar), 3.70 (1.5 H, s, CCH₂Ar), 3.79 (1.5 H, t, J=4.8 Hz, CH₂OCH₃), 3.81 (0.5 H, t, J=5.0 Hz, CH₂OCH₃), 4.16 (1.5 H, t, J=4.8 Hz, CH₂CH₂OCH₃), 4.19 (0.5 H, t, J=5.0 Hz, CH₂CH₂OCH₃), 6.94 (1.5 H, d, J=9.0 Hz, H-3′,5′), 6.98 (0.5 H, d, J=8.5 Hz, H-3′,5′), 7.25 -7.42 (12 H, m, ArH). ¹³C NMR ppm (CDCl₃) 13.20* (C-4), 24.57, 24.67* (C-3), 58.98, 59.01* (OCH₃), 67.08*, 67.14 (CH₂OCH₃), 70.91, 70.94* (CH₂CH₂OCH₃), 114.08, 114.16* (C-3′,5′), 125.70*, 126.13, 126.08*, 126.24 (C-4″,4′″), 127.95*, 128.00, 128.20*, 128.56*, 129.13*, 130.20*, 130.23 (ArC—H), 135.65*, 135.72, 138.40*, 138.49, 138.91, 138.93*, 140.55, 140.57*, 143.31, 143.35* (C-1,2,1′,1′,1′″), 157.21* (C-4′). m/z 372 (M⁺, 100%), 313 (58), 59 (28).

4-{2-Ethyl-3-[4-(2-methoxyethylamino)-phenyl]-3-phenylallyl}-phenol

IRvₘₐₓ (film) 3387 (OH), 1610, 1513 (C=C) cm-⁻¹. ¹ H NMR δ (CDCl₃) 1.05 (3 H, t, J=7.3 Hz, H-4), 2.49 (2 H, q, J=7.4 Hz, H-3), 3.30 (2 H, t, J=5.3 Hz, NCH₂), 3.41 (OCH₃), 3.42 (2 H, s, CCH₂Ar), 3.64 (2 H, t, J=5.0 Hz, OCH₂CH₂OCH₃), 6.59 (2 H, d, J=8.6 Hz, H-3′,5′), 6.75-7.35 (11 H, m, Ar—H). ¹³C NMR ppm (CDCl₃) 13.28 (C-4), 24.52 (C-3), 36.43 (CCH₂Ar), 43.46 (NCH₂), 58.71 (OCH₃), 71.00 (OCH₂CH₂OCH₃), 112.69 (C-3′,5′), 127.97 (C-4″), 129.29, 129.69, 129.69, 130.24, 130.47 (C-2′,6′,2″,3″,5″,6″,62′″,3′″,5′″,6′″), 132.77 (C-1′), 138.22, 140.65, 146.42, 148.13, 148.19 (C-1,2,1″,1′″,4′), 153.69 (C-4′″).

m/z 374 (M⁺+1, 100%), 329 (14), 129 (3).

N′-[4-(2-Benzyl-1-phenylbut-1-enyl)-phenyl]-N,N-dimethylethane-1,2-diamine

IRvₘₐₓ (film) 3372 (NH), 1603, 1507 (C=C) cm-⁻¹. ¹ H NMR δ (CDCl₃) 1.01 (3 H, t, J=7.5 Hz, H-4), 2.10 (2 H, q, J=7.5 Hz, H-3), 2.24 (6 H, s, N(CH₃)₂), 2.45 (2 H, t, J=6.8 Hz, NCH₂CH₂N(CH₃)₂), 3.57 (2 H, s, CCH₂Ar), 3.84 (2 H, t, J=6.8 Hz, NCH₂CH₂N(CH₃)₂), 7.17-7.38 (14 H, m, ArH). ¹³C NMR ppm (CDCl₃) 13.18 (C-4), 24.90 (C-3), 37.09 (CCH₂Ar), 45.48 (N(CH₃)₂), 49.20 (NCH₂CH₂N(CH₃)₂), 55.63 (NCH₂CH₂N(CH₃)₂), 125.96, 126.55 (C-4″,4′″.), 128.08 (C-3′,5′), 128.21, 128.38, 128.57, 129.21, 130.04 (ArC—H), 137.04, 138.19, 140.01, 140.18, 142.45, 143.72 (C-1,2,1′,1″,1′″,4′).

385 (M⁺+1, 100%), 72 (15), 58 (25)

Pharmacological Tests

The following tests were carried out to determine the activity of the compounds of the invention. The compounds have potent anti-proliferative effects with desirable cytotoxicity profiles in human breast tumour MCF-7 cell lines in vitro. Such compounds therefore have potential in estrogen related diseases and as antiosteoporotic compounds.

The following examples describe the assays carried out.

EXAMPLE 14—Determination of the antiproliferative effects on human MCF-7 cell lines.

MTT is a yellow tetrazolium salt which is taken up only by metabolically active cells and subsequently cleaved by mitochondrial dehydrogenases to yield a purple crystalline formazan dye. On solubilisation this purple color may be read spectrophotometrically at 570 nm. The absorbance measured at this wavelength is directly proportional to the amount of viable cells present.

Procedure : The human breast tumor MCF-7 cell line was cultured in Eagles minimum essential medium in a 5% CO₂ atmosphere with 10% fetal calf serum. The medium was supplemented with 1% non-essential amino acids. The cells were trypsinized and seeded at a density of 1.5×10⁴ into a 96-well plate and incubated at 37° C., 5% CO₂ atmosphere for 24 h. After this time they were treated with 2 μl volumes of test compound which had been pre-prepared as stock solutions in ethanol to furnish the concentration range of study, 1 nM-100 μM, and re-incubated for a further 72 h. Control wells contained the equivalent volume of the vehicle ethanol (1% v/v). The culture medium was then removed and the cells washed with 100 μl PBS and 50 μl MTT added, to reach a final concentration of 1 mg/ml MTT added. The cells were incubated for 2 h in darkness at 370° C. At this point solubilisation was begun through the addition of 200 μl DMSO and the cells maintained at room temperature in darkness for 20 min to ensure thorough color diffusion before reading the absorbance. The absorbance value of control cells (no added compound) was set to 100% cell viability. Graphs of absorbance versus cell density per well were prepared to assess cell viability. Table 1 shows the results of percentage cell viability versus the concentration of two test compounds 4 and 21A. These results are at least comparable to results achieved using known estrogen receptor antagonists such as tamoxifen.

TABLE 1

| Concentration | 1 nM | 10 nM | 100 nM | 1 μM | 10 μM | 50 μM | 100 μM |
|---|---|---|---|---|---|---|---|
| % Cell viability | 103.8 ± | 100.3 ± | 91.14 ± | 85.13 ± | 29.11 ± | 1.58 ± | 1.27 ± |
| Compound 4 | 3.16 | 1.27 | 3.48 | 6.65 | 2.21 | 0.63 | 0.31 |
| % Cell viability | 88.3 ± | 69.9 ± | 55.1 ± | 40.6 ± | 3.2 ± | 12.6 ± | ND |
| Compound 21A | 6.9 | 2.2 | 7.1 | 5.7 | 2.0 | 6.3 | |
| % Cell viability | 84.9 ± | 92.5 ± | 65.1 ± | 45.06 ± | 23.4 ± | 2.3 ± | ND |
| Tamoxifen | 9.8 | 4.2 | 5.8 | 5.8 | 13.6 | 1.6 | |
| control | | | | | | | |

EXAMPLE 15—Determination of the cytotoxic effects on human MCF-7 cell lines.

Lactate dehydrogenase (LDH) is a cytosolic enzyme released upon cell lysis (death). Through the use of a commercial LDH assay kit, released LDH converts a substrate tetrazolium salt into a soluble red formazan product. The absorbance of this dye can be measured directly at 490 nm, the color formed is proportional to the number of lysed cells, and as such the extent of cytotoxic activity for the compound added may be assessed.

Procedure : As with the cell proliferation assay, human MCF-7 breast cancer cells were plated at a density of 1.5×10⁴ per well in a 96-well plate, then incubated at 37° C., 5% CO₂ atmosphere for 24 h. The cells were treated with the compound of choice at varying concentrations (1nM-100 μM), then incubated for a further 72 h. Following incubation 50 μl aliquots of medium were removed to a fresh 96-well plate. A 50 μl per well LDH substrate mixture was added and the plate left in darkness at room temperature for 30 minutes. Stop solution (50 μl) was added to all wells before reading the absorbance at 490 nm. A control of 100% lysis was determined for a set of untreated cells which were lysed through the addition of 20 μl lysis solution to the media 45 min prior to harvesting. Table 2 shows the percentage cell lysis versus the concentration test compounds 4 and 21A. These results are at least comparable to results achieved using known estrogen receptor antagonists such as tamoxifen.

TABLE 2

| Con- | | 10 | 100 | | | | 100 |
|---|---|---|---|---|---|---|---|
| centration | 1 nM | nM | nM | 1 μM | 10 μM | 50 μM | μM |
| % | 3.67 ± | ND | ND | ND | 34.76 ± | 49.44 ± | 49.14 ± |
| Cytotoxicity | 0.53 | | | | 1.80 | 2.85 | 1.90 |
| Compound 4 | | | | | | | |
| % | ND | ND | ND | ND | 41.9 ± | 32.8 ± | ND |
| Cytotoxicity | | | | | 1.2 | 3.8 | |
| Compound | | | | | | | |
| 21A | | | | | | | |
| % | ND | ND | ND | ND | 49.2 ± | 38.2 ± | ND |
| Cytotoxicity | | | | | 3.1 | 6.1 | |
| Tamoxifen | | | | | | | |
| control | | | | | | | |

EXAMPLE 16—Determination of the binding affinity of compounds for estrogen receptor

Binding affinity (Ki value) for the estrogen receptor is measured by the ability of the compound to displace tritium-labelled estradiol from the receptor site.

Procedure Estrogen receptor-rich cytosol was obtained from the uteri of humanely sacrificed Sprague-Dawley immature rats (100-150 g mass). Briefly, the uteri were homogenized in 0.01 M sodium phosphate buffer, pH 7.3, containing 0.15 M NaCl, 0.1% gelatin and 0.01% sodium azide. The homogenate was then centrifuged at 100,000×g, 40° C. The cytosol thus isolated was pretreated with dextran coated charcoal (DCC on ice) [13] and re-isolated using centrifugation, before freezing at−20° C. for later use. The protein concentration of cytosol samples was determined using a standard Bradford protein assay and an appropriate protein concentration range (150 μg protein in a total volume of 0.14 ml) for assay prepared. The required amounts of tritium-labeled (hot) and non-labeled (cold) estradiol were calculated using standard saturation curve techniques. A fresh buffer solution was prepared (Tris[tris(hydroxymethyl)-aminomethane]buffer-10 mM, pH 7.4, containing 1.5 mM EDTA and 3 mM sodium azide). Displacement testing of the compounds was facilitated through the incubation of a buffered solution of a known concentration of the test compound with hot estradiol solution (specific activity 157 Ci/mmol-final conc. 5 nM/ tube), followed by the addition of estrogen receptor-rich cytosol (150 μg protein). Total and non-specific binding control assays were determined in the absence and presence of 14 μl of a 0.2 mM cold estradiol solution respectively, properly corrected for the presence of ethanol in the test (displacement) samples. Samples were vortexed to ensure homogeneity and refrigerated at 40° C. for 16-20 h. After this time the samples were retreated with DCC on ice and centrifuged for ten min at 3500×g. A 170 μl sample was pipetted from each vial and diluted with 10 ml scintillation fluid (Ecoscint). A scintillation control containing 28 μl of 5 nM hot estradiol in 10 ml scintillation fluid was also prepared to facilitate theoretical activity calculations. The samples thus prepared were counted for radioactivity by liquid scintillation counting. Binding values were obtained as counts per minute (cpm) and were converted to disintegrations per minute (dpm) and computationally analysed using sigmoidal curve fitting programs EBDA and LIGAND [14] to fit the displacement curves and to calculate binding affinity values (Ki) for the test compounds.

Results:

The following table illustrates the relative binding affinity values measured for selected compounds, based on their ability to displace radiolabelled estradiol from estrogen receptor-rich cytosol. Values are expressed as the mean determined±SEM. Some of these compounds have binding affinity values comparable to binding values for known estrogen receptor antagonists.

| Compound | Ki |
|---|---|
| 1 | 459 ± 230 nM |
| 6 | 503 ± 98 nM |
| 14 | 2.39 ± 0.35 μM |
| 20 | 2.48 ± 0.45 μM |

EXAMPLE 17—Assay for determining apoptotic induction by compounds

Fluorescence Activated Cell Sorting

Procedure Following treatment of MCF-7 cells with apoptotic compounds, the cells were washed three times with PBS before being trypsinised and centrifuged at 300 g for 5 min. They were then resuspended in 200 μl PBS, made up to 2 ml with ice-cold ethanol (70% v/v) and left to sit on ice for at least 1 h to fix them. Approximately 1 h prior to use they were centrifuged at 300 g for 3 min and the supernatant carefully pipeted off. The pellet was resuspended in 800 μl PBS. RNAase (100 μL; 1mg/mL) and 100 μL of the fluorescent dye propidium iodide (PI; 400 μg/mL) which binds DNA were added. The tubes were vortexed and incubated at 37° C. for 30 min. Flow cytometry was performed with a FACS calibur flow cytometer from Becton Dickinson. FACS data was analysed using the programme Cell Quest.

Results:

The following table shows the induction of apoptosis in MCF-7 cells in response to compound 25. MCF-7 cells were treated with either vehicle (1% ethanol v/v or 50 μM compound 10 for 16 hours. Cells were then prepared for FACS analysis. Changes in the distribution of cell cycle phases G1, S and G2/M as well as the sub-G1 apoptotic peak are indicated. FACS analysis was performed with propidium iodide stained cells. The results obtained are comparable to results achieved using known estrogen receptor antagonists.

% Cell cycle phases

| Treatment | sub-G1 | G1 | S | G2/M |
|---|---|---|---|---|
| Control | 1.5 | 71.5 | 3.0 | 24.0 |
| 25 | 20.1 | 51.3 | 7.6 | 21.0 |

The effect of the compounds on endometrial and osteoblastic cell lines for assignment of SERM status may be carried out using assays known in the art, preferably an alkaline phosphatase assay.

The ability of the compounds to stimulate uterine cell growth may be assessed by an alkaline phosphatase assay in human endometrial Ishikawa and in SaOs-2 osteoblast cells respectively, as described previously [15].

It will be appreciated that the compounds may have useful pharmacological properties other than those described above.

Appendices 1 and 2 show IC₅₀ data for representative compounds presented as the mean of triplicate determinations using a standard enzymatic MTT inhibition of proliferation assay technique.

The invention is not limited to the embodiments hereinbefore described which may be varied in detail.

APPENDIX 1

| Generic | Compd. | | R₂ (where | R₃ (where | IC₅₀ Range | Mass Spec |
|---|---|---|---|---|---|---|
| Type | ID | R₁ | applicable) | applicable) | (μM) | M/Z (M⁺⁾ |
| I | 1 | OCH₂CH₂N(Me)₂ | — | — | 10-15 | 385 |
| I | 2 | OCH₂CH₂N | — | — | 10-15 | 413 |
| | | (Et)₂ | | | | |
| I | 3 | OCH₂CH₂ | — | — | 1-5 | 411 |
| | | (pyrrolidinyl) | | | | |
| I | 4 | OCH₂CH₂ | — | — | 1-5 | 425 |
| | | (piperidyl) | | | | |
| I | 5 | OCH₂CH₂ | — | — | 40-50 | 427 |
| | | (morpholinyl) | | | | |
| II | 6 | OCH₂CH₂N(Me)₂ | — | — | 20-25 | 385 |
| II | 7 | OCH₂CH₂N(Et)₂ | — | — | 15-20 | 413 |
| II | 8 | OCH₂CH₂ | — | — | 10-15 | 411 |
| | | (pyrrolidinyl) | | | | |
| II | 9 | OCH₂CH₂ | — | — | 70-80 | 425 |
| | | (piperidyl) | | | | |
| II | 10 | OCH₂CH₂ | — | — | 1-5 | 427 |
| | | (morpholinyl) | | | | |
| III | 11 | OCH₂CH₂N(Me)₂ | — | — | 115-120 | 385 |
| III | 12 | OCH₂CH₂N(Et)₂ | — | — | 20-25 | 413 |
| III | 13 | OCH₂CH₂ | — | — | 20-25 | 411 |
| | | (pyrrolidinyl) | | | | |
| III | 14 | OCH₂CH₂ | — | — | 15-20 | 425 |
| | | (piperidyl) | | | | |
| III | 15 | OCH₂CH₂ | — | — | 50-55 | 427 |
| | | (morpholinyl) | | | | |
| IV | 16 | OCH₂CH₂N(Me)₂ | — | — | 25-30 | 385 |
| IV | 17 | OCH₂CH₂N(Et)₂ | — | — | 55-60 | 413 |
| IV | 18 | OCH₂CH₂ | — | — | 140-145 | 411 |
| | | (pyrrolidinyl) | | | | |
| IV | 19 | OCH₂CH₂ | — | — | 65-70 | 425 |
| | | (piperidyl) | | | | |
| IV | 20 | OCH₂CH₂ | — | — | 15-20 | 427 |
| | | (morpholinyl) | | | | |
| V | 21 | OCH₂CH₂ | p-OPiv | H | 1-10 | 512 |
| | | (pyrrolidinyl) | | | | (M + 1) |
| V | 21A | OCH₂CH₂ | m-OPiv | H | 0.25 | 512 |
| | | (pyrrolidinyl) | | | | (M + 1) |
| V | 22 | OCH₂CH₂N(Me)₂ | p-OPiv | H | 10-50 | 486 |
| | | | | | | (M + 1) |
| V | 23 | OCH₂CH₂ | p-OPiv | H | 0.1-1 | 528 |
| | | (morpholinyl) | | | | (M + 1) |
| V | 24 | OCH₂CH₂N(Me)₂ | p-OH | H | 0.01-0.1 | 402 |
| | | | | | | (M + 1) |
| V | 25 | OCH₂CH₂ | p-OH | H | 0.01-0.1 | 428 |
| | | (pyrrolidinyl) | | | | (M + 1) |
| V | 26 | OCH₂CH₂ | p-OPiv | o-OMe | 1-10 | 542 |
| | | (pyrrolidinyl) | | | | (M + 1) |

APPENDIX 2

| Generic | Compd. | | R₂ (where | R₃ (where | IC₅₀ | Mass Spec |
|---|---|---|---|---|---|---|
| Type | ID | R₁ | applicable) | applicable) | (10⁻⁶M) | (M⁺) |
| VII | 27 | NH(CH₂)₂OMe | — | — | 33.7 | 372 (M + 1) |
| VII | 28 | NH(CH₂)₂OC₂H₅ | — | — | 36.8 | 386 (M + 1) |
| VII | 29 | NH(CH₂)₂OC₃H₇ | — | — | 46.9 | 400 (M + 1) |
| VII | 30 | NH(CH₂)₃OMe | — | — | 35.4 | 386 (M + 1) |
| VII | 31 | NH(CH₂)₃OC₂H₅ | — | — | 14.1 | 400 (M + 1) |
| VII | 32 | NH(CH₂)₃OC₃H₇ | — | — | 23.3 | 414 (M + 1) |
| VII | 33 | NH(CH₂)₂OH | — | — | 39.5 | 358 |
| VII | 34 | NH(CH₂)₃OH | — | — | 34.0 | 372 |
| VII | 35 | O(CH₂)₂OH | — | — | 24.9 | 357 |
| VII | 36 | O(CH₂)₃OH | — | — | 20.2 | 371 |
| VII | 37 | NH(CH₂)₂OMe | p-OMe | H | 34.4 | 401 |
| VII | 38 | NH(CH₂)₂OMe | p-OMe | p-OMe | 51.8 | 431 |
| VII | 39 | NH(CH₂)₂OMe | H | p-OMe | 49.5 | 401 |
| VII | 40 | O(CH₂)₂OMe | — | — | 41.7 | 372 |
| VII | 41 | NH(CH₂)₂OMe | — | p-OH | 32.4 | 387 |
| VII | 42 | NH(CH₂)₂N(CH₃) | — | — | 13.7 | 384 |

### REFERENCES

1. Katzenellenbogen, B.S. Estrogen receptors : bioactivities and interactions with cell signalling pathways. Biol. Reprod. 1996, 54, 287-293.

2. Beato, M.; Sanchez-Pacheco, A. Interaction of steroid hormone receptors with the transcription initiation complex. Endocr. Rev. 1996, 17, 587-609.

3. Lerner, L. J.; Jordan, V. C. Development of antiestrogens and their use in breast cancer: Eighth Cain Memorial Award Lecture. Cancer Res. 1990, 50, 4177-4189.

4. Feng W, Ribeiro R C J, Wagner R L, Nguyen H, Apriletti J W, Fletterick R J, Baxter J D, Kushner P J, West B L: Hormone dependant coactivator binding to a hydrophobic cleft on nuclear receptors. Science (1998) 280:1747-1749.

5. Kuiper GGJM, Enmark E, Pelto-Huikko M, Nilsson S, Gustafsson J A: Cloning of a novel estrogen receptor expressed in rat prostrate and ovary. Proc Natl Acad Sci USA (1996) 93:5925-5930.

6. Maruyama K, Endoh H, Sasaki-Iwaoka H, Kanou H, Shimaya E, Hashimoto S, Kato S, Kawashima H: A novel isoform of the rat estrogen receptor beta with 18 amino acid insertion in the ligand binding domain as a, putative dominant regulator of estrogen action. Biochem Biophys Res Comm (1998) 246:142-147.

7. Gustafsson J-A: Estrogen receptor-β-a new dimension in estrogen mechanism of action. J Endocinol (1999) 163: 379-383.

8. Budtz PE: Role of proliferation and apoptosis in net growth rates of human breast cancer cells (MCF-7) treated with oestradiol and/or tamoxifen. Cell Proliferat (1999) 32:289-302.

9. Johnston S R D, Boeddinghaus I M, Riddler S, Haynes B P, Hardcastle I R, Rowlands M, Grimshaw R, Jarman M, Dowsett M: Idoxifene antagonises estradiol-dependent MCF-7 breast cancer xenograft growth through sustained induction of apoptosis. Cancer Res (1999) 59: 3646-3651.

10. Lloyd, D. G.; Meegan, M. J. Recent advances in estrogen receptor antagonists. IDrugs 2000, 3, (6) 632-642 & Refs therein.

11. Bradbury, S. P.; Mekenyan, O. G.; Ankley, G. T. The role of ligand flexibility in predicting biological activity : Structure-activity relationships for aryl hydrocarbons, estrogen and androgen receptor binding affinity. Environ. Toxicol. Chem.1998, 17, 15-25.

12. Balant, L. P., Doelker, E. Metabolic considerations in prodrug design. In: Wolff, M. E. (Ed.) Burger's Medicinal Chemistry and Drug Discovery. 1994, 5th edition, Vol. 1: Principles & Practice. Wiley: New York -pp. 949-977 & ref.s therein.

13. Fishman, J. H. Stabilization of estradiol-receptor complexes by elimination of cytosolic factors. Biophys. Res. Commun. 1983, 110, 713-718.

14. Munson, P. J.; Rodbard, D. LIGAND: a versatile computerised approach for the characterisation of ligand binding systems. Anal Biochem. 1980,107, 220-239.

15. Littlefield, B. A., Gurpide, E., Markiewicz, L., McKinley, B. and Hochberg, R. B. A simple and sensitive microtiter plate estrogen bioassay based on stimulation of alkaline phosphatase in Ishikawa cells: estrogenic action ofadrenal steroids. Endocrinol. 1990 127, 2757-2762.

1. A compound of the formula I 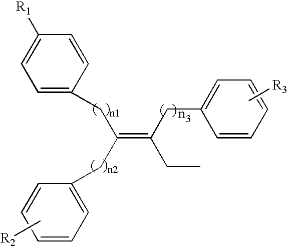
wherein R₁=H, OH, Br, NH₂ or R₄ wherein R₄ is O(CH₂)₂NRₐR_{b} or NH(CH₂)ₓNRₐR_{b} or NH(CH₂)ₓRₐ-R_{b} or O(CH₂)ₓRₐ-R_{b} and Rₐ and R_{b} are independently H, O, CH₃, C₂ H₅, C₃ H₇ or optionally part of a heterocyclic ring system of the structure: 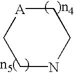
wherein n₄ and n₅ are independently 0 or 1 and both are not 0, and
A is CH₂ or O;
and x is 2 or 3,
R₂ is independently one of H, OH, OPiv, OAc, OCONHMe, OMe R₃ is independently one of H, OH, OPiv OMe or para O(CH₂)₂NRₐR_{b} wherein Rₐ and R_{b} are as defined above,
n₁, n₂ and n₃₌₀ or 1 independently, and n₁, n₂ and n₃ are such that only one n=1 at any one time where n₁, n₂ and n₃ are not all equal to 0,
and isomers, prodrugs and pharmaceutically acceptable salts thereof.

2. A compound as claimed in claim 1 wherein R₂≠R₃

3. A compound as claimed in claim 1 wherein R₁ is O(CH₂)₂Rₐ-R_{b} and Rₐ and R_{b} are as defined in claim 1.

4. A compound as claimed in claim 3 wherein n₃ is 1 and Rₐ-R_{b} are selected from pyrrolidinyl or piperidyl.

5. A compound as claimed in claim 1 or 2 having the formula 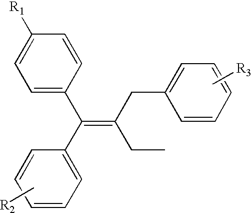
wherein
R₁ is as in claim 1,
R₂ is independently one of H, OH, OPiv, OAc, OCONHMe, OMe
R₃ is independently one of H, OH, OPiv, OMe,
and isomers, prodrugs and pharmaceutically acceptable salts thereof.

6. A compound as claimed in claim 5 wherein at least one or both of R₂ or R₃ contains an oxygen group.

7. A compound as claimed in claim 5 or 6 wherein R₂ or R₃ are in any position on the associated ring.

8. A compound as claimed in claim 5 or 6 wherein R₂ is a para hydroxy group.

9. A compound as claimed in claim 8 wherein R₃ is hydrogen.

10. A compound as claimed in claim 5 or 6 wherein R₂ is an ester group in the para position.

11. A compound as claimed in claim 10 wherein R₃ is hydrogen.

12. A compound as claimed in claim 10 wherein R₃ is an ortho methoxy group.

13. A compound as claimed in claim 1 or 2 having the formula 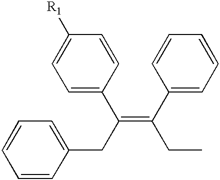
wherein R₁=H, OH, Br, NH₂ or R₄ wherein R₄ is H, O(CH₂)₂NRₐR_{b} or NH(CH₂)ₓNRₐR_{b} or NH(CH₂)ₓRₐ-R_{b} or O(CH₂)ₓRₐ-R_{b} and Rₐ and R_{b} are independently H, 0, CH₃ , C₂ H₅, C₃ H₇ or optionally part of a heterocyclic ring system of the structure: 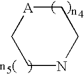
wherein n₄ and n₅ are independently 0 or 1 and both are not 0,
A is CH₂ or O,
and x is 2 or 3,
and isomers, prodrugs and pharmaceutically acceptable salts thereof.

14. A compound as claimed in claim 13 wherein R₁ is O(CH₂)₂Rₐ-R_{b} and Rₐ-R_{b} is morpholinyl.

15. A compound as claimed in claim 13 wherein R₁ is O(CH₂)₂Rₐ-R_{b} and Rₐ-R_{b} is pyrrolidinyl.

16. A compound as claimed in claim 1 or 2 having the formula 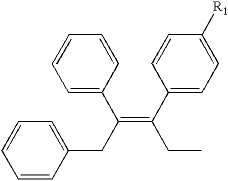
wherein R₁=H, OH, Br, NH₂ or R₄ wherein R₄ is H, O(CH₂)₂NRₐR_{b} or NH(CH₂)ₓNRₐR_{b} or NH(CH₂)ₓRₐ-R_{b} or O(CH₂)ₓRₐ-R_{b}and Rₐ and R_{b} are independently H, O, CH₃, C₂ H₅, C₃ H₇ or optionally part of a heterocyclic ring system of the structure: 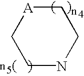
wherein n₄ and n₅ are independently 0 or 1 and both are not 0,
A is H₂or O,
and x is 2 or 3,
and isomers, prodrugs and pharmaceutically acceptable salts thereof.

17. A compound as claimed in claim 1 or 2 having the formula 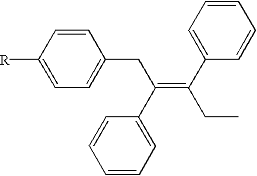
wherein R=H, OH, Br, NH₂ or R₄ wherein R₄ is O(CH₂)₂NRₐR_{b} or NH(CH₂)ₓNRₐR_{b} or NH(CH₂)ₓRₐ-R_{b} or O(CH₂)ₓRₐ-R_{b} and Rₐ and R_{b} are independently H, O, CH₃ , C₂ H₅, C₃ H₇,or optionally part of a heterocyclic ring system of the structure: 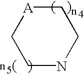
wherein n₄ and n₅ are independently 0 or 1 and both are not 0,
A is CH₂ or O,
and x is 2 or 3,
and isomers, prodrugs and pharmaceutically acceptable salts thereof.

18. A compound as claimed in claim 1 or 2 having the formula 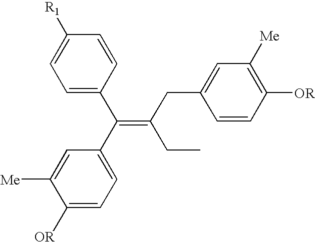
wherein R=H, Me or Piv and wherein R₁=H, Br, NH₂ or R₄ wherein R₄ is O(CH₂)₂NRₐR_{b} or NH(CH₂)ₓNRₐR_{b} or NH(CH₂)ₓRₐ-R_{b} or O(CH₂)ₓRₐ-R_{b} and Rₐ and R_{b} are independently H, O, CH₃, C₂ H₅, C₃ H₇ or optionally part of a heterocyclic ring system of the structure: 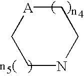
wherein n₄ and n₅ are independently 0 or 1 and both are not 0,
A is CH₂ or O,
and x is 2 or 3,
and isomers, prodrugs and pharmaceutically acceptable salts thereof.

19. A compound selected from
2-Benzyl-1-phenyl-1-[4-(dimethylaminoethoxy)phenyl]but-1-ene
2-Benzyl-1-phenyl-1-[4-(diethylaminoethoxy)phenyl]but-1-ene
2-Benzyl-1-phenyl-1-[4-(pyrrolidinylethoxy)phenyl]but-1-ene
2-Benzyl-1-phenyl-1-[4-(pipyridinylethoxy)phenyl]but-1-ene
2-Benzyl-1-phenyl-1-[4-(morpholinylethoxy)phenyl]but-1-ene
1-Benzyl-2-phenyl-[(4-dimethyleaminoethoxy)phenyl]but-1-ene
1-Benzyl-2-phenyl-[1-(4-diethylaminoethoxy)phenyl]-but-1-ene
1-Benzyl-2-phenyl-[1-(4-pyrrolidinylethoxy)phenyl]-but-1-ene
1-Benzyl-2-phenyl-[1-(4-pipyridinylethoxy)phenyl]-but-1-ene
1-Benzyl-2-phenyl[1-(5-morpholinylethoxy)phenyl]but-1-ene
1-Benzyl-1-phenyl-2-[(4-dimethylaminoethoxy)phenyl]but-1-ene
1-Benzyl-1-phenyl-2-[(4-diethylaminoethoxy)phenyl]but-1-ene
1-Benzyl-1-phenyl-2-[(4-pyrrolidinylethoxy)phenyl]but-1-ene
1-Benzyl-1-phenyl-2-[(4-pipyridinylethoxy)phenyl]but-1-ene
1-Benzyl-1-phenyl-2-[(4-morpholinylethoxy)phenyl]but-1-ene
1,2-Diphenyl-2-[2-(dimethylaminoethoxy)benzyl]but-1-ene
1,2-Diphenyl-2-[2-(diethylaminoethoxy)benzyl]but-1-ene
1,2-Diphenyl-2-[(4-pyrrolidinylethoxy)benzyl]but-1-ene
1,2-Diphenyl-2-[(4-pipyridinylethoxy)benzyl]but-1-ene
1,2-Diphenyl-2-[(4-morpholinylethoxy)benzyl]but-1-ene
2,2-Dimethyl-propionic acid 4-{2-benzyl-1-[4-(2-pyrrolidin-1-yl-ethoxy-phenyl]-but-1-enyl}-phenyl ester
2,2-Dimethyl-propionic acid 4-{2-benzyl-1-[4-(2-dimethylamino-ethoxy-phenyl]-but-1-enyl}-phenyl ester
2,2-Dimethyl-propionic acid 4-{2-benzyl-1-[4-(2-morpholin-4-yl -ethoxy-phenyl]-but-1-enyl}-phenyl ester
4-{2-Benzyl-1-[4-(2-dimethylamino-ethoxy)-phenyl]-but-1-enyl}-phenol
4-{2-Benzyl-1-[4-(2-pyrrolidin-1-yl-ethoxy)-phenyl]-but-1-enyl}-phenol
2,2-Dimethyl-propionic acid 4-{2-(2-methoxybenzyl)-1-[4-(2-pyrrolidin-1 -yl-ethoxy)-phenyl]-but-1-enyl}-phenyl ester
[4-(2-Benzyl-1-phenylbut-1-enyl)-phenyl]-(2-methoxyethyl)-amine
[4-(2-Benzyl-1-phenylbut-1-enyl)-phenyl]-(2-ethoxyethyl)-amine
[4-(2-Benzyl-1-phenylbut-1-enyl)-phenyl]-(2-prop oxyethyl)-amine
[4-(2-Benzyl-1-phenylbut-1-enyl)-phenyl]-(3-methoxypropyl)-amine
[4-(2-Benzyl-1-phenylbut-1-enyl)-phenyl]-(3-ethoxypropyl)-amine
[4-(2-Benzyl-1-phenylbut-1-enyl)-phenyl]-(3-propoxypropyl)-amine
2-[4-(2-Benzyl-1-phenylbut-1-enyl)-phenylamino]-ethanol
3-[4-(2-Benzyl-1-phenylbut-1-enyl)-phenylamino]-propan-1-ol
2-[4-(2-Benzyl-1-phenylbut-1-enyl)-phenoxy]-ethanol
3-[4-(2-Benzyl-1-phenylbut-1-enyl)-phenoxy]-propan-1-ol
{4-[2-Benzyl-1-(4-methoxyphenyl)-but-1-enyl]-phenyl}-(2-methoxyethyl)-amine
{4-[2-(4-methoxybenzyl)-1-(4-methoxyphenyl)-but-1-enyl]-phenyl}-(2-methoxyethyl)-amine
{4-[2-(4-methoxybenzyl)-1-phenylbut-1 -enyl]-phenyl} -(2-methoxyethyl)-amine
1-(2-Benzyl-1-phenylbut-1-enyl)-4-(2-methoxyethoxy)-benzene
4-{2-Ethyl-3-[4-(2-methoxyethylamino)-phenyl]-3-phenylallyl}-phenol
N′-[4-(2-Benzyl-1-phenylbut-1-enyl)-phenyl]-N,N-dimethylethane-1,2-diamine.

20. 2-Benzyl-1-phenyl-1-[4-(pyrrolidinylethoxy)phenyl]but-1-ene.

21. 2-Benzyl-1-phenyl-1-[4-(pipyridinylethoxy)phenyl]but-1 -ene.

22. 1-Benzyl-2-phenyl[1-(5-morpholinylethoxy)phenyl]but-1-ene.

23. 2,2-Dimethyl-propionic acid 4-{2-benzyl-1-[4-(2-pyrrolidin-1-yl-ethoxy-phenyl]-but-1-enyl}-phenyl ester.

24. 4-{2-Benzyl-1-[4-(2-dimethylamino-ethoxy)-phenyl]-but-1-enyl}-phenol.

25. 4-{2-Benzyl-1-[4-(2-pyrrolidin-1-yl-ethoxy)-phenyl]-but-1-enyl}-phenol.

26. [4-(2-Benzyl-1-phenylbut-1-enyl)-phenyl]-(3-ethoxypropyl)-amine.

27. 3-[4-(2-Benzyl-1-phenylbut-1-enyl)-phenoxy]-propan-1-ol.

28. N′-[4-(2-Benzyl-1-phenylbut-1-enyl)-phenyl]-N,N-dimethylethane-1,2-diamine.

29. A compound as claimed in any preceding claim wherein the compound is antiosteoporotic.

30. A compound claimed in any of claims 1 to 28 for inhibiting the proliferation of and/or induction of apoptosis in human breast cancer cells.

31. A compound as claimed in any of claims 1 to 28 for modulating the estrogen receptor(s).

32. A compound selected from
4-(2-benzyl-1-phenylbut-1-enyl)phenyl-amine
N-[4-(2-benzyl-1-phenylbut-1 -enyl)-phenyl]-2,2,2-trifluoroacetamide
N-[4-(2-benzyl-1-phenylbut-1-enyl)-phenyl]-2,2,2-trifluoro-N-[3-(tetrahydropyran-2-yloxy)-propyl]-acetamide
[4-(2-Benzyl-1-phenylbut-1-enyl)-phenyl]-[3-(tetrahydropyran-2-yloxy)-propyl]-amine
[4-(2-benzyl-1-phenylbut-1-enyl)-phenyl]-[3-(tetrahydropyran-2-yloxy)-propyl]-carbamic acid ethyl ester
[4-(2-benzyl-1-phenylbut-1-enyl)-phenyl]-(3-hydroxypropyl)-carbamic acid ethyl ester.

33. A compound substantially as hereinbefore described with reference to the examples.

34. A pharmaceutical composition comprising a compound as claimed in any of claims 1 to 31.

35. A pharmaceutical composition comprising a compound as claimed in any of claims 1 to 31 in combination with a pharmaceutically acceptable carrier or diluent.

36. A pharmaceutical composition comprising a compound as claimed in any of claims 1 to 31 in combination with a pharmaceutically active compound.

37. A composition as claimed in claim 36 wherein the pharmaceutically active compound is an anti-cancer drug.

38. A composition as claimed in claim 37 wherein the anti-cancer drug is cisplatin.

39. A pharmaceutical composition as claimed in any of claims 34 to 38 for administration in the form of an emulsion, liposome, patch, powder and/or complex tablet, capsule, syrup, dose-metered inhaler.

40. A pharmaceutical composition as claimed in any of claims 34 to 39 in a form for oral, intravenous, intramuscular, intraperitoneal, intradermal, intravesicular and/or rectal administration.

41. Use of a compound as claimed in any of claims 1 to 31 in the preparation of a medicament for the prophylaxis and/or treatment of estrogen related conditions and/or conditions where the induction of apoptosis is desirable.

42. Use as claimed in claim 41 wherein the condition is any one or more of obesity, hormone dependent breast cancer, osteoporosis, estrogen deficiency, arthritis, cardiovascular disease, ovarian cancer, artherosclerosis, colon tumor, endometriosis, Alzheimer′s disease, non-insulin dependent (type II) diabetes, infertility, prostrate tumor, melanoma, acne, hypercholesterolemia, CNS disease, contraception, conditions related to hair follicles, macular degeneration, urinary incontinence, estrogen receptor-expressing and estrogen receptor-expressing tumors, leukaemia.

43. Use of a compound as claimed in any of claims 1 to 31 in inhibiting the proliferation of and/or induction of apoptosis in breast cancer cells.

44. A method for the treatment and/or prophylaxis of an estrogen related disease comprising administering an effective amount of a compound as claimed an any of claims 1 to 31 or a composition as claimed in any of claims 34 to 43.
